(19) United States
(12) Patent Application Publication
(10) Pub. No.: US 2026/0080987 A1
(43) Pub. Date: Mar. 19, 2026

(54) SYSTEM FOR CENTRALIZED MANAGEMENT OF ELECTRONIC DENTAL RECORDS

(71) Applicant: MyEDR Solutions LLC, Huntington Station, NY (US)

(72) Inventors: Daniel Vinoi, Centereach, NY (US); Alexis Davidson, Manorville, NY (US)

(73) Assignee: MyEDR Solutions LLC, Huntington Station, NY (US)

(21) Appl. No.: 19/226,362

(22) Filed: Jun. 3, 2025

Related U.S. Application Data
(63) Continuation of application No. 18/595,881, filed on Mar. 5, 2024, now abandoned.

(51) Int. Cl. G16H 10/60; G16H 40/20

(52) U.S. Cl. CPC G16H 10/60; G16H 40/20

(57) ABSTRACT

In an embodiment, a system configured to maintain a patient account associated with a dental patient is disclosed. The system is further configured to cause a user interface to be presented on a patient device associated with the dental patient and to receive, from the patient device, an indication corresponding to a digital file associated with the dental patient that was generated by a first dental provider. The system is further configured to obtain the digital file based on the received indication, generate an electronic dental record (EDR) file based at least in part on the obtained digital file, store the EDR file in an EDR database, associate the EDR file with the patient account of the dental patient, receive information identifying a second dental provider from the patient device, and provide the second dental provider with access to the EDR file associated with the patient account.

### RELATED APPLICATIONS

This application is a continuation of U.S. patent application Ser. No. 18/595,881 filed Mar. 5, 2024, entitled “System For Centralized Management Of Electronic Dental Records,” the disclosure of which is hereby incorporated by reference in its entirety.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### BACKGROUND

This application relates to patient control of electronic dental records, and in particular, to systems and methods for maintaining and managing the use of electronic dental records under the control of a patient.

Patient dental records, such as x-rays or other scans are typically generated, held and stored by each individual dental practice that performs dental work on a patient. Sharing of patient dental records between dental practices is often difficult or impossible. In addition, patient access and control of their dental records often relies solely on the dental provider that generated these records. If a patient uses multiple dentists or decides to change dentists, the new dentist often has to develop a new set of patient dental records for the patient, for example, by taking additional x-rays or other associated examinations, which can be detrimental to the patient.

### SUMMARY

A new system for centralized management of electronic dental records is disclosed. The system comprises a processor coupled to memory, in which the processor is configured to maintain a patient account associated with a dental patient. The processor is further configured to cause a user interface to be presented on a patient device associated with the dental patient, and receive, from the patient device, an indication corresponding to a digital file associated with the dental patient that was generated by a first dental provider. The processor is further configured to obtain, based on the received indication, the digital file, generate an electronic dental record (EDR) file based at least in part on the obtained digital file, store the EDR file in an EDR database, and associate the EDR file with the patient account of the dental patient. The processor is further configured to receive, from the patient device, information identifying a second dental provider, and provide the second dental provider with access to the EDR file associated with the patient account.

The indication corresponding to the digital file may comprise information associated with an email account associated with the dental patient. The digital file may be obtained from the email received by the email account, for example, from an attachment of the email. The processor may be further configured to receive a request to set up the patient account from the patient device. The processor may be further configured to set up the patient account based at least in part on the request, wherein the setting up of the patient account comprises assigning the email account to the patient account.

The indication corresponding to the digital file may comprise a location of the digital file on the patient device. Obtaining the digital file may comprise uploading the digital file from the location of the digital file on the patient device. The indication corresponding to the digital file may comprise an indication of the performance of an image capture operation by the patient device. Obtaining the digital file may comprise obtaining the digital file from the image capture operation performed by the patient device.

Providing the second dental provider with access to the EDR file associated with the patient account may comprise attaching the EDR file associated with the patient account to an email, or generating a link to the EDR file associated with the patient account, wherein the link is usable by the second dental provider to view the EDR file associated with the patient account.

The processor may be further configured to identify a plurality of dental providers that meet a set of criteria corresponding to the dental patient, wherein the plurality of dental providers comprises the second dental provider. The processor may be further configured to request estimates for dental services from the identified dental providers, obtain estimates from at least one of the identified dental providers including an estimate from the second dental provider, cause presentation of the obtained estimates on the user interface of the patient device, and obtain, from the patient device, a selection of the estimate obtained from the second dental provider.

Receiving information identifying the second dental provider from the patient device may comprise receiving the set of criteria from the patient device. Providing the second dental provider with access to the EDR file associated with the patient account may be based at least in part on the request of the estimate for dental services from the second dental provider. The processor may be further configured to obtain an indication of a schedule of available appointments corresponding to the second dental provider, cause the user interface to present the indication on the patient device, obtain, from the patient device, a selection of an available appointment from the schedule, and cause a communication with a provider device associated with the second dental provider that indicates that the dental patient has selected the available appointment from the schedule.

A new method for centralized management of electronic dental records is disclosed. The method is performed by a processor comprising hardware. The method comprises maintaining a patient account associated with a dental patient and causing a user interface to be presented on a patient device associated with the dental patient. The method further comprises receiving, from the patient device, an indication corresponding to a digital file associated with the dental patient that was generated by a first dental provider, obtaining the digital file based on the received indication, generating an electronic dental record (EDR) file based at least in part on the obtained digital file, and storing the EDR file in an EDR database. The method further comprises associating the EDR file with the patient account of the dental patient, receiving, from the patient device, information identifying a second dental provider, and providing the second dental provider with access to the EDR file associated with the patient account.

The indication corresponding to the digital file may comprise information associated with an email account associated with the dental patient. The digital file may be obtained from the email received by the email account. The method may further comprise receiving a request to set up the patient account from the patient device and, based on the request, setting up the patient account and assigning the email account to the patient account. Providing the second dental provider with access to the EDR file associated with the patient account may comprise attaching the EDR file associated with the patient account to an email.

The method may further comprise identifying a plurality of dental providers that meet a set of criteria corresponding to the dental patient, the plurality of dental providers comprising the second dental provider. The method may further comprise requesting estimates for dental services from the identified dental providers, obtaining estimates from at least one of the identified dental providers including an estimate from the second dental provider, causing presentation of the obtained estimates on the user interface of the patient device, and obtaining, from the patient device, a selection of the estimate obtained from the second dental provider. Receiving information identifying the second dental provider may comprise receiving the set of criteria from the patient device. Providing the second dental provider with access to the EDR file associated with the patient account may be based at least in part on the request of the estimate for dental services from the second dental provider.

A new non-transitory computer-readable medium for centralized management of electronic dental records is disclosed. The non-transitory computer-readable medium stores instructions that, when executed by a processor, cause the processor to maintain a patient account associated with a dental patient and cause a user interface to be presented on a patient device associated with the dental patient. The instructions further cause the processor to receive, from the patient device, an indication corresponding to a digital file associated with the dental patient that was generated by a first dental provider. The instructions further cause the processor to obtain the digital file based on the received indication, generate an electronic dental record (EDR) file based at least in part on the obtained digital file, store the EDR file in an EDR database, associate the EDR file with the patient account of the dental patient, receive, from the patient device, information identifying a second dental provider, and provide the second dental provider with access to the EDR file associated with the patient account.

The indication corresponding to the digital file may comprise information associated with an email account associated with the dental patient. The digital file may be obtained from the email received by the email account. Providing the second dental provider with access to the EDR file associated with the patient account may comprise attaching the EDR file associated with the patient account to an email.

The foregoing summary is illustrative only and is not intended to be in any way limiting. These and other illustrative embodiments include, without limitation, apparatus, systems, methods and computer-readable storage media. In addition to the illustrative aspects, embodiments, and features described above, further aspects, embodiments, and features will become apparent by reference to the drawings and the following detailed description.

### BRIEF DESCRIPTION OF THE DRAWINGS

The invention is illustrated in the figures of the accompanying drawings which are meant to be exemplary and not limiting, in which like references are intended to refer to like or corresponding parts.

FIG. 1 is a block diagram of a system according to an embodiment.

FIG. 2 is a block diagram of an example patient portal module of the system of FIG. 1 according to an embodiment.

FIG. 3 is a block diagram of an example electronic dental record (EDR) capture module of the system of FIG. 1 according to an embodiment.

FIG. 4 is a block diagram of an example EDR export module of the system of FIG. 1 according to an embodiment.

FIG. 5 is a block diagram of an example provider recommendation and estimate module of the system of FIG. 1 according to an embodiment.

FIGS. 6-17 are illustrations of an example user interface according to an embodiment.

FIGS. 18-28 are illustrations of an example user interface according to an embodiment.

FIG. 29 is a flow diagram of an example process according to an embodiment.

FIGS. 30A-30G are flow diagrams of example processes according to an embodiment.

FIG. 31 is a flow diagram of an example email integration process according to an embodiment.

FIG. 32 is a flow diagram of an example email import process according to an embodiment.

FIG. 33 is a flow diagram of an example treatment plan process according to an embodiment.

FIG. 34 is a flow diagram of an example add provider process according to an embodiment.

FIG. 35 is a flow diagram of an example provider search process according to an embodiment.

FIG. 36 is a flow diagram of an example appointment and estimate request process according to an embodiment.

FIG. 37 is a flow diagram of an example add insurance process according to an embodiment.

### DETAILED DESCRIPTION OF THE INVENTION

Subject matter will now be described more fully hereinafter with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. It is to be understood that other embodiments may be utilized and structural changes may be made without departing from the scope of the illustrative embodiments. Likewise, a reasonably broad scope for claimed or covered subject matter is intended. Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of exemplary embodiments in whole or in part. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

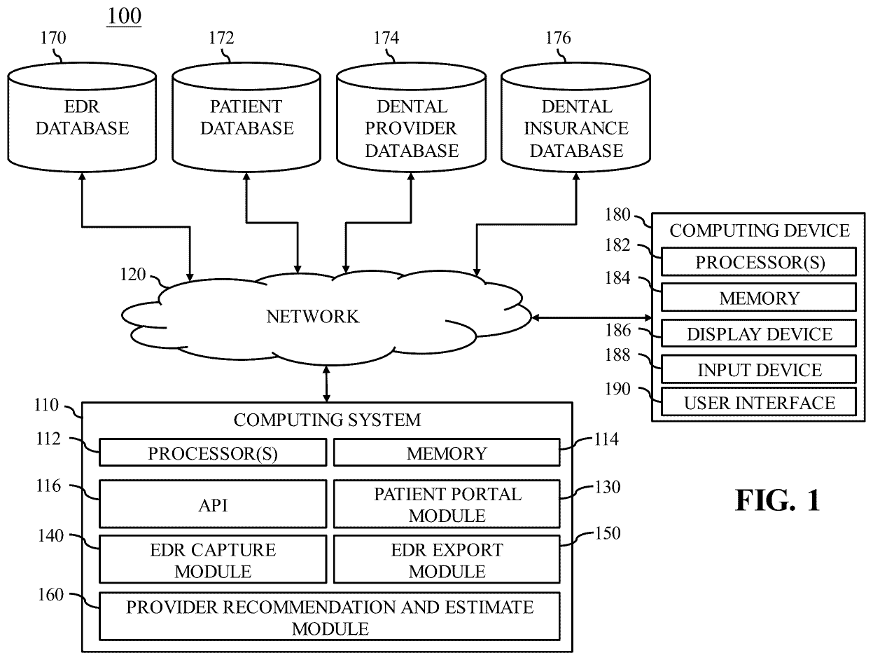

With reference to FIGS. 1-5, a system 100 is disclosed that is configured to empower patients to manage, store and control access to their electronic dental record (EDR). System 100 comprises a computing system 110, network 120, EDR database 170, patient database 172, dental provider database 174, dental insurance database 176 and one or more computing device(s) 180. In other embodiments, system 100 may comprise additional or alternative components including additional computing systems 110, databases 170-176, or computing devices 180 or alternative components for any of the components. In some embodiments, databases 170-176 may comprise data stored on separate storage devices or storage systems while in other embodiments one or more of databases 170-176 may be stored on the same storage device or storage system. In some embodiments, for example, since EDR database 170 comprises patient medical information such as, e.g., dental records, EDR database 170 may be stored separately or segregated from databases 172, 174 and 176 or in any other manner needed to comply with data privacy requirements such as those found in the Health Insurance Portability and Accountability Act (HIPAA) and the Health Information Technology for Economic and Clinical Health (HITECH) Act.

Computing system 110 comprises one or more processors 112, memory 114, an application programming interface (API) 116, a patient portal module 130, an EDR capture module 140, an EDR export module 150 and a provider recommendation and estimate module 160.

Processor(s) 112 may comprise, e.g., a processor, a microprocessor, a microcontroller, an application-specific integrated circuit (ASIC), a field-programmable gate array (FPGA), a graphics processing unit (GPU), a printed circuit board (PCB) or any other type of processing circuitry, as well as portions or combinations of such circuitry elements.

Memory 114 may comprise, e.g., random access memory (RAM), read-only memory (ROM), flash memory or other types of memory, in any combination. The memory and other memories disclosed herein should be viewed as illustrative examples of what are more generally referred to as “processor-readable storage media” that may store executable program code of one or more software programs.

API 116 comprises functionality that interfaces between computing system 110, databases 170-176 and computing device 180. API 116 may also provide an interface between processor 112, memory 114 and modules 130-160.

Network 120 is configured to connect computing system 110, databases 170-176 and computing device 180 and comprises one or more wired, wireless or combined wired/wireless networks and corresponding hardware such as hubs, switches, access points, network interfaces or other hardware commonly found in a network. Example wired and wireless networks that may be utilized include the Internet, a wide area network (WAN), a local area network (LAN), satellite, telephone, cable, a fiber-optic, cellular, ethernet, WiFi, WiMAX, Bluetooth®, any other network or connection or any combination thereof.

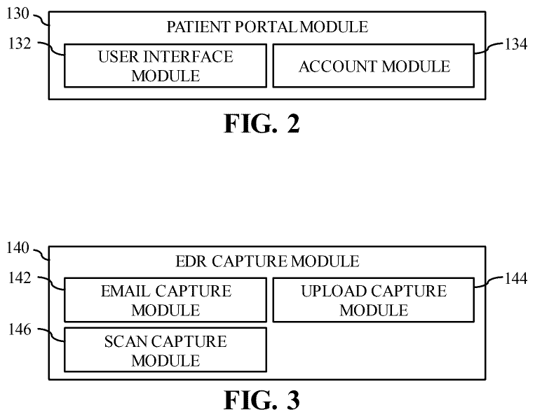

With reference to FIGS. 1 and 2, patient portal module 130 is configured to generate an interactive patient portal for presentation on computing device 180. Patient portal module 130 comprises a user interface module 132 and an account module 134.

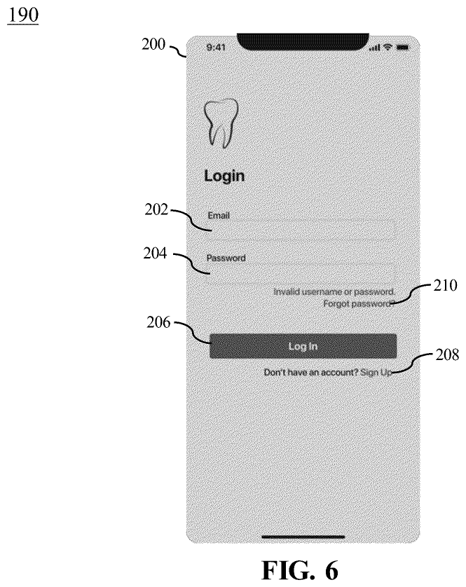

User interface module 132 is configured to generate a user interface 190 that is presented by the interactive patient portal on display device 186 of computing device 180, e.g., as part of a web page, mobile application or in any other manner via API 116. Example illustrations of user interface 190 are shown in FIGS. 6-28 and will be described in more detail below.

Account module 134 is configured to manage patient accounts including login information, dental provider information, insurance information or any other information relevant to the patient. As an example, account module 134 may receive login information from the patient, e.g., via the interactive patient portal, and compare the login information to account information found in patient database 172.

New patients may be requested by account module 134 to create a new account and provide account information such as, e.g., a name, date of birth, contact email address, dental provider information, insurance information or any other relevant data to assist the patient in logging into the interactive patient portal or for providing services to the patient via interactive patient portal. Some or all of the account information may be scanned in by a patient, e.g., using a camera, scanner or in any other manner, and parsed or processed to generate the relevant account information. As an example, an image of an insurance card may be received by account module 134 from the patient's mobile phone and may be parsed or image processed to determine insurance information. The patient may also or alternatively enter any account information manually, e.g., in fields of user interface 190.

The patient account information is stored by account module 134 in patient database 172. In a case where the patient provides information about a dental provider that they are currently using or would like to use, a link between the patient account and the dental provider may be generated, e.g., between the patient account information stored in patient database 172 and dental provider information stored in dental provider database 174. In a case where the dental provider is not already present in dental provider database 174, the dental provider may be added to the dental provider database 174 and, where needed, additional information about the dental provider may be obtained by system 100. Similarly, in a case where the patient provides information about a dental insurance that they are currently using or would like to use, a link between the patient account and the dental insurance may be generated, e.g., between the patient account information stored in patient database 172 and dental insurance information stored in dental insurance database 176. In a case where the dental insurance is not already present in dental insurance database 176, the dental insurance may be added to the dental insurance database 176 and, where needed, additional information about the dental insurance may be obtained by system 100, e.g., from the corresponding dental insurance provider.

In some embodiments, account module 134 may be configured to generate a patient email address based on the patient account information that is configured for use with system 100, e.g., for obtaining EDRs from dental providers, providing EDRs to dental providers or any other use. As an example, in some cases, EDRs may have large files sizes which may not be transferable via a personal email account of the patient. The patient email address provided by account module 134 may be associated with the patient account in patient database 172 and configured to handle the large file sizes that are common with EDRs which may facilitate a smooth transfer of the EDR files to and from system 100 for storage and retrieval from EDR database 170 in association with the patient account.

With reference to FIGS. 1 and 3, EDR capture module 140 is configured to obtain and capture patient EDRs for storage in EDR database 170 in association with a particular patient account. EDR capture module 140 comprises an email capture module 142, an upload capture module 144 and a scan capture module 146.

Email capture module 142 is configured to parse through and download or otherwise obtain EDR files from an email address. For example, the patient may have received EDR files from a dental provider on their own email address or on the patient email address provided by system 100. As an example, the patient may provide EDR capture module 142 with access to their email account, e.g., a personal email account, business email account or any other email account at which the user has received emailed EDR files from a dental provider. Email capture module 142 is configured to monitor and parse through the emails and email attachments, identify EDR files, and download any identified EDR files for storage in EDR database 170.

Upload capture module 144 is configured to obtain files provided by the patient via an upload mechanism. Example upload mechanisms may include file transfer technologies such as, e.g., File Transfer Protocol (FTP), Secure File Transfer Protocol (SFTP), Secure Shell (SSH) File Transfer Protocol (SFTP), Simple File Transfer Protocol (SFTP), Managed File Transfer (MFT), a web-based file transfer technology or any other file transfer technology. As an example, the patient may drag and drop an EDR file onto a user interface element of user interface 190, browse to a file location and select an EDR file, or otherwise indicate to upload capture module 144 a location of an EDR file for upload. Upload capture module 144 is configured to obtain the EDR file from the location and store the EDR file in EDR database 170 in association with the patient account using any of the above-mentioned file transfer technologies or any other file transfer technologies.

In some embodiments, a dental provider may also utilize system 100 and have an account with system 100. In such a case, the patient may be able to authorize the dental provider, e.g., via user interface 190 of the patient portal, to upload EDR files directly from the dental provider's database for storage in EDR database 170 in association with the patient's account in patient database 172, e.g., using any of the above file transfer technologies or any other file transfer technology. In some embodiments, EDR files uploaded by the dental provider may be made available to the patient for approval before associating the uploaded EDR files with the patient account.

Scan capture module 146 is configured to obtain files via a scanner or other image capture device and generate a corresponding EDR. As an example, the patient may connect scan capture module 146 to a scanner, insert one or more printouts of dental images or records into the scanner, and instruct scan capture module 146 to obtain the one or more dental images or records from the scanner. Scan capture module 146 may then command the scanner to scan the one or more dental images or records, or otherwise obtain the scanned one or more dental images or records from the scanner.

In another embodiment, user interface 190 may comprise a view associated with scan capture module 146 that is configured to enable an image capture function on a device being used by the patient, e.g., a camera on a mobile device. Scan capture module 144 may utilize the image capture function of the device to obtain dental images or records, e.g., by taking a picture or otherwise scanning the dental images or records using the image capture function of the device.

Scan capture module 146 is configured to generate one or more EDR files based on the obtained one or more dental images or records. As an example, scan capture module 146 may be configured to combine multiple scanned dental images or records into a single or multiple EDR files, e.g., depending on the content of each dental image or record. In some embodiments, scan capture module 146 may be configured to perform image processing, AI pattern matching or any other action on the scanned dental images or records to determine how to store the scanned dental images or records in one or more EDR files. In some embodiments, scan capture module 146 may be configured to parse metadata associated with the scanned dental image or records to determine how to store the scanned dental images or records in one or more EDR files. The EDR files may be stored by scan capture module 146 in EDR database 170 and associated with the patient's account in patient database 174.

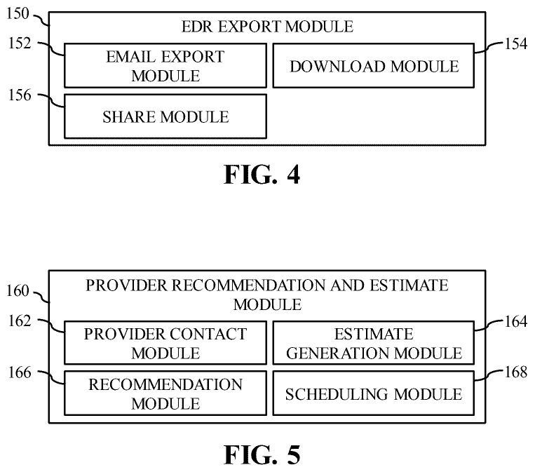

With reference to FIGS. 1 and 4, EDR export module 150 is configured to allow a patient to export EDR files, e.g., for personal storage outside of system 100 or to a dental provider. EDR export module 150 comprises an email export module 152, a download module 154 and a share module 156.

Email export module 152 is configured to enable the patient to generate an email attachment comprising EDR files or links to EDR files for sending to another party such as, e.g., a dental provider. In a case where system 100 provides the user with an email address, email export module 152 may be configured to automatically generate an email based on provided information such as, e.g., recipient email address, recipient name or title, selected EDR files or any other information. In some embodiments, email export module 152 may also or alternatively be configured to automatically generate and send the email when requested. In some embodiments, the email may be a secured and encrypted email such that only a recipient of the email may know its contents. In a case where links to EDR files are provided, encryption and authentication mechanisms may be implemented such as, e.g., requiring a login and 2-factor authentication based on the recipient email address in order to access the files. Any other security features may also or alternatively be implemented.

Download module 154 is configured to enable a patient to download one or more EDR files directly to a computing device, e.g., using any of the file transfer technologies described above. In some embodiments, download module 154 may be utilized by a dental provider to download patient EDR files directly to a dental provider computing device or server, e.g., for use in conjunction with providing dental services or an estimate of dental services to the patient.

Share module 156 is configured to enable a patient to share or otherwise link their one or more of their EDR files to a dental provider or any other individual or entity that has an account on system 100. As an example, the patient may select a dental provider that is has an account with system 100, request to share EDR files with that dental provider, select the EDR files to be shared with that dental provider, and cause a sharing of the EDR files with that dental provider e.g., via user interface 190 of the patient portal. In some embodiments, the patient may select to share access to one or more of their EDR files stored in EDR database 170 with a user or entity that does not have an account with system 100. In such a case, system 100 may provide temporary access, e.g., via a link, may request that the user or entity set up an account to have access, or provide access to the shared documents in any other manner. Appropriate verification or security measures may be taken to ensure that such access is protected and safe.

With reference to FIGS. 1 and 5, provider recommendation and estimate module 160 is configured to recommend dental providers to patients and generate estimates for dental work from dental providers for the patients. Provider recommendation and estimate module 160 comprises a provider contact module 162, estimate generation module 164, a recommendation module 166 and a scheduling module 168.

Provider contact module 162 is configured to contact dental provider, e.g., based on information contained in one or more of patient database 172, dental provider database 174 and insurance database 176, to obtain estimates for dental work to be performed on the patient. As an example, provider contact module 162 may be configured to determine a dental insurance of the patient, e.g., using one or both of patient database 172 and insurance database 176 and identify providers that accept the dental insurance, e.g., using dental provider database 174. For example, the patient may need a dental provider located within a certain geographic area, having a particular specialty and that takes their dental insurance, e.g., as participating in-network provider or as partial payment out-of-network. In some embodiments, the patient may specify whether or not to include only participating in-network providers or both in-network and out-of-network providers. These and any other criteria may be obtained by provider contact module 162, e.g., from the patient or identified by provider contact module 162 based on the patient account, patient EDR files or any other patient related information, to identify a list of potential dental providers for the desired service.

Provider contact module 162 is configured to engage with dental providers to obtain estimates for the desired dental service, e.g., based on the list of potential dental providers. As an example, provider contact module 162 may be configured to contact a dental provider based on contact information for that dental provider found in dental provider database 174. In an example, provider contact module 162 may be configured to email the dental provider at an email address associated with that dental provider in dental provider database 174. The email may comprise a request for quote or estimate for the patient and any other relevant information needed to make a quote. In some embodiments, EDR files or other patient information may be withheld from the dental provider until the dental provider confirms that they are interested in providing an estimate for the desired service, e.g., to inhibit unnecessary exposure and transfer of EDR files and other patient information to dental providers that are not interested in providing an estimate. As an example, the email request for quote or estimate may comprise a link, activatable element or other feature that allows the dental provider to indicate interest in providing an estimate.

In some embodiments, alternative contact information such as, e.g., a phone number for the dental provider, may be utilized by provider contact module 162. For example, provider contact module 162 may comprise an AI system that is configured to make phone calls, send text messages, send social media messages or any other messages to dental providers to solicit estimates for desired dental services and update information for dental providers in dental provider database 174. In some embodiments, the AI phone call may solicit an indication that the dental provider would like to provide an estimate and obtain appropriate contact or other information for transferring or providing access to any relevant EDR files and patient information needed for the purposes of an estimate. In some embodiments, the AI phone call may also obtain an estimate from the dental provider.

The email or other contact may also seek to confirm with the dental provider whether or not the dental provider accepts the patient's dental insurance. As an example, the email may comprise an activatable element or link that may be selected by the dental provider to indicate that the patient's dental insurance is accepted and whether it is accepted in-network or out-of-network. Similarly, the AI phone call or other contact may also confirm with the dental provider whether the patient's insurance is accepted and whether it is accepted in-network or out-of-network.

Relevant EDR files and other patient information may be released to the dental provider for the purposes of generating and providing an estimate, e.g., using email, sharing or any of the file transfer technologies as described above for EDR export module 150. As mentioned above, in some embodiments, the relevant EDR files and patient information may only be released after receiving an indication that the dental provider is interested in providing an estimate. In other embodiments, the relevant EDR files and other patient information may be released along with the request for quote or estimate.

In some embodiments, a dental provider that has an account with server 100 may receive an indication that an estimate has been requested, e.g., via contact information for the dental provider stored in dental provider database 174, via a push notification or in any other manner. In such a case, the dental provider may be provided with access to the relevant patient EDR files and information for the purposes of making the estimate without the need to obtain/download the files, ensuring that the files remain safely stored on system 100. The dental provider may then submit the estimate via system 100 to estimate generation module 164, e.g., by filling out a form or in any other manner.

Estimate generation module 164 is configured to receive estimates from dental providers based on the contact made by provider contact module 162. In some embodiments, provider contact module 162 may provide the dental provider with a fillable form, with a link to a fillable form, or a link to a location for uploading an estimate. Estimate generation module 164 is configured to obtain the estimate from the dental provider and generate an estimate output for presenting to the patient, e.g., via user interface 190 of the patient portal. In some embodiments, the estimate may also comprise a recommendation on a type of service to be performed by the dental provider. As an example, the dental provider may, after reviewing the EDR files and patient information, determine that certain services are recommended and provide an estimate for the same to estimate generation module 164. For example, different dental providers may provide different recommendations and estimates for a variety of different dental services based on their own review of the patient's EDR files and information.

Estimate generation module 164 may be configured to collate all recommendations and estimates from dental providers as is or, in some embodiments, may be configured to separate the recommendations and estimates into groupings associated with particular recommended treatments and estimates so that a patient may be enabled to compare the prices for similar recommended treatments. In some embodiments, the dental providers may be requested to provide separate or itemized estimates for each recommended treatment to enable a comparison of estimates for the same recommended treatment by generation module 164 and by the patient.

Recommendation module 166 is configured to provide a list comparing recommended treatments and estimates to the patient, e.g., via user interface 190. As an example, recommendation module 166 may provide the comparison in a format that allows the patient to browse through and filter by recommendations, recommended treatments, estimate price, dental provider, service location or any other criteria to enable the patient to select the desired dental provider and treatment.

Scheduling module 168 is configured to enable a patient to schedule an appointment with a dental provider. As an example, the patient may select a dental provider or recommended treatment from the list provided by recommendation module 166 or in any other manner, e.g., by reviewing a list of local dental providers generated from dental provider database 174.

In some embodiments, scheduling module 168 may initiate contact with a dental provider to determine availability for appointments, e.g., via e-mail, text, phone or in another manner, and provide the patient with an indication of the dental provider's available slots for selection of an appointment, e.g., a calendar or other representation. As an example, scheduling module 168 may utilize the AI phone call to contact the dental provider identify the availability for appointments and then provide the patient with an indication of the available appointments, e.g., via the representation such as a calendar. In other embodiments, scheduling module 168 may have access to provider availability via system 100 where, for example, if the dental provider has an account with system 100, their schedule may also be stored or linked to system 100 for use by scheduling module 168 in determining an availability of the dental provider. Scheduling module 168 may then allow the patient to select an available slot in the dental provider's schedule for their appointment.

In some embodiments, scheduling module 168 may interface with 3^{rd} party scheduling tools to both determine the availability of the dental provider and schedule the appointment for the patient. As an example, 3^{rd} party scheduling tools such as that provided by Zocdoc® or another 3^{rd} party for scheduling appointments with a dental provider may be utilized to schedule the appointment. In some embodiments, scheduling module 168 may redirect the patient to the 3^{rd} party scheduling tool, e.g., to a Zocdoc® page associated with the dental provider, for scheduling the appointment. As part of the redirect, scheduling module 168 may be configured to automatically fill in some or all of any relevant or necessary information for scheduling the appointment as needed for the patient. In other embodiments, scheduling module 168 may interface with a backend server of the 3^{rd} party scheduling tool to present the patient with an embedded scheduling tool, e.g., calendar or another too, in user interface 190 that may leverage scheduling data, functionality or other features of the 3^{rd} party scheduling tool for assisting the patient in scheduling an appointment for the selected dental provider and recommended treatment. In some embodiments, API 116 may be utilized by scheduling module 168 to interface with the 3^{rd} party scheduling tool.

In some embodiments, computing system 110 utilizes a machine learning algorithm to implement some or all of modules 130-160. The machine learning algorithm may include, for example, decision tree learning, association rule learning, artificial neural networks, deep learning, inductive logic programming, support vector machine (SVM), Bayesian networks, rule-based machine learning, another type of machine learning, or any combination thereof. As an example, in some embodiments the machine learning algorithm may be trained using data obtained from databases 170-176, e.g., using EDR data, patient data, dental provider data, dental insurance data or other data, to implement the functionality of patient portal module 130, EDR capture module 140, EDR export module 150 and provider recommendation and estimate module 160.

Databases 170-176 comprise data storage devices or data storage systems that are configured to store data associated with EDRs, patients, dental providers and dental insurance that may be utilized by computing system 110.

Example data storage devices that may be utilized by databases 170-176 include hard disk drives (HDD), solid state drives (SSDs) or other storage technologies. In some embodiments, the data storage devices may be implemented using non-volatile memory (NVM) devices such as flash memory. Other types of NVM devices that can be used to implement at least a portion of the data storage devices include non-volatile random access memory (NVRAM), phase-change RAM (PC-RAM) and magnetic RAM (MRAM). These and various combinations of multiple different types of NVM devices may also be used. The particular storage devices used may be varied in other embodiments, and multiple distinct storage device types may be used within a data storage system. The term “storage device” as used herein is intended to be broadly construed, so as to encompass, for example, flash drives, solid state drives, hard disk drives, hybrid drives or other types of storage devices.

Example data storage systems that may be utilized by databases 170-176 include network-attached storage (NAS), storage area networks (SANs), direct-attached storage (DAS) and distributed DAS, as well as combinations of these and other storage types, including software-defined storage. Other types of data storage systems that can be used including all-flash and hybrid flash storage arrays, software-defined storage systems, cloud storage systems, object-based storage systems, and scale-out NAS clusters and associated accelerators. Combinations of multiple ones of these and other data storage systems can also be used in implementing databases 170-176 in an illustrative embodiment.

In some embodiments, one or more of databases 170-176 may be combined into a single database, or one or more databases 170-176 may be stored together in the same storage system.

EDR database 170 is configured to store patient EDR files and other sensitive patient information associated with the patient EDR files including, e.g., 2d and 3d scans, treatment plans, procedure summaries, implant types, materials used, allergies, bloodwork or any other information about the patient that may be stored in conjunction with patient EDR files and used for diagnosis or treatment of the patient. The 2d and 3d scans may include radiographs (sometimes referred to as x-rays) of various parts of the patient's mouth such as bite wing, periapical, panoramic or any other radiographs commonly taken by dental providers, e.g., as part of a mouth survey or series. The 2d and 3d scans may include advanced imaging such as, e.g., computed tomography (CT) scans, Magnetic Resonance Imaging (MRI) scans or any other type of advanced imaging scans. The 2d or 3d scans may include dental photography such as extra-oral, intra-oral, mini intra-oral or any other photographic imaging. The 2d or 3d scans may include digital impression images that may be utilized for 3d printing or other purposes. One example digital impression image format may include (Stereolithography) STL files although any other digital impression image format may alternatively be utilized.

Patient database 172 is configured to store patient details associated with a patient's account including, for example, username, login information, links to or associations with particular EDR files in EDR database 170, links to or associations with particular dental providers in dental provider database 174, links to or associations with particular dental insurance information in dental insurance database 176 or any other information about the patient that is utilized by system 100.

Dental provider database 174 is configured to store information about dental providers. As an example, details about each dental provider that has an account with system 100 may be stored in dental provider database 174 and may be utilized to link the dental provider to patient EDR files, e.g., in a case where a patient has shared or otherwise granted access to one or more of their files to that dental provider for treatment or estimate purposes. Dental provider database 174 may also store details about unaffiliated dental providers for use in providing recommendations and estimates to the patients. As an example, each dental provider may have a profile stored in dental provider database, whether or not they have an account with system 100. The profile may comprise contact information, treatment locations, accepted insurance or any other information about the dental provider and may comprise an indication of whether or not the dental provider has an account with system 100. In some embodiments, the accepted insurance may comprise a link or connection to a corresponding insurance policy found in dental insurance database 176.

The information about each dental provider may be provided by the dental provider, e.g., during account creation, may be provided by the patient, e.g., during patient account creation or during a session where the patient adds an association to the dental provider to their account, may be obtained from the dental provider by EDR capture module 140, may be obtained from the dental provider by provider recommendation and estimate module 160 or may be obtained from the dental provider in any other manner. In some embodiments, for example, information about a dental provider may be obtained by emails, AI phone calls or in any other manner and stored in dental provider database.

Dental insurance database 176 may comprise information or details about various dental insurance policies that may be available to patients, that patients have signed up for either directly or through an employer, or in any other manner. Each dental insurance policy in dental insurance database may be linked to one or more patients and dental providers. As an example, a patient profile in patient database 172 may comprise a link to the dental insurance policy information stored in dental insurance database for that patient. The dental insurance policy may also comprise links to dental providers that accept that dental insurance policy. Dental insurance database 176 may be populated by dental insurance information provided by patients, dental providers, directly from dental insurance providers or in any other manner. As an example, dental insurance database 176 may be linked to dental insurance providers and may act as a marketplace for the dental insurance providers such that patients may be offered with options for obtaining dental insurance via server 100 if desired. Once a dental insurance is linked to the patients account, dental insurance database 176 enables patients to quickly identify those dental providers that accept their dental insurance policy via links between that dental insurance and the dental providers found in dental provider database 174.

Computing device 180 comprises one or more processors 182, memory 184, a display device 186, an input interface 188 and a user interface 190. In some embodiments, for example, computing device 180 comprises a personal computer, smart phone, tablet, smart watch, or any other device.

Processor(s) 182 comprise, e.g., a processor, a microprocessor, a microcontroller, an application-specific integrated circuit (ASIC), a field-programmable gate array (FPGA), a graphics processing unit (GPU), a printed circuit board (PCB) or any other type of processing circuitry, as well as portions or combinations of such circuitry elements.

Memory 184 comprises, e.g., random access memory (RAM), read-only memory (ROM), flash memory or other types of memory, in any combination. The memory and other memories disclosed herein should be viewed as illustrative examples of what are more generally referred to as “processor-readable storage media” storing executable program code of one or more software programs.

Display device 186 comprises, e.g., a screen, a monitor, a television, phone, smart device, or any other device that is configured to present data or images to a user.

Input device 188 comprises, e.g., a keyboard, mouse, touch screen, or any other physical interface that is configured to receive a user input from a user.

With reference to FIGS. 1 and 6-28, user interface 190 comprises an interactive graphical user interface that may be presented to a user, e.g., via display device 186. The user may interact with user interface 190 using input device 188 to cause an execution of the functionality of computing system 110 via network 120.

With reference to FIG. 6, user interface 190 presents a patient with a view 200 of the interactive patient portal for logging into system 100. View 200 comprises fields 202 and 204 for entering username and password information, a log in element 206 that is selectable by the patient to initiate a login process with server 100 and a sign up element 208 that is selectable by the patient to initiate an account setup process. While shown as email for field 202, any other username may also or alternatively be utilized. Any other fields or activatable elements that are common to login processes may also or alternatively be present including, e.g., a forgot password element 210 or any other activatable element. In some embodiments, fields 202 and 204 may be replaced with other mechanisms for authenticating a user including, e.g., fingerprint scanning, retinal scanning, multi-factor authentication, barcodes such as, e.g., QR codes, or any other security mechanism that may be utilized to enable a patient to log into their account on system 100. Upon receiving the patient's login credentials, account module 134 is configured to verify the patient and grant or deny access to the patient account, for example, as described above.

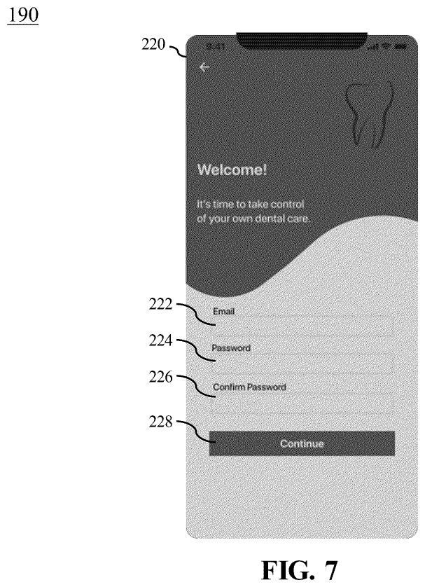

With reference to FIG. 7, if the patient selects sign up element 208 to initiate the account set up process, user interface 190 presents the patient with a view 220. View 220 comprises an email field 222, password field 224, confirm password field 226 and continue element 228. The patient may, for example, enter their login credentials such as email and password in fields 222, 224 and 226 and confirm their information by selecting confirm element 228 to set up the account. In some embodiments, view 220 may also or alternatively provide the capability to establish other modes of authentication including, e.g., fingerprint scanning, retinal scanning, facial recognition, multi-factor authentication or any other manner of authentication.

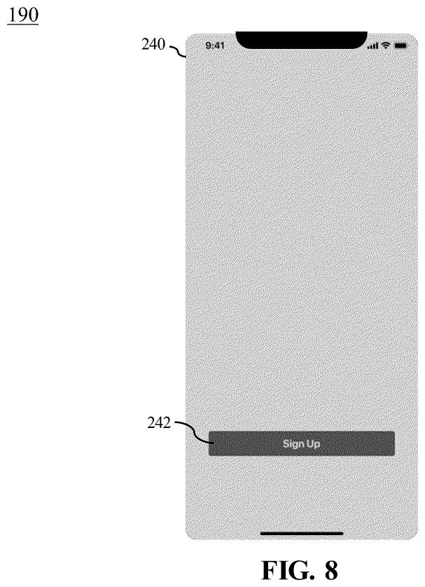

With reference to FIG. 8, user interface 190 presents the patient with a view 240. View 240 comprises a sign up element 242 that is selectable by the patient to sign up for an account with system 100.

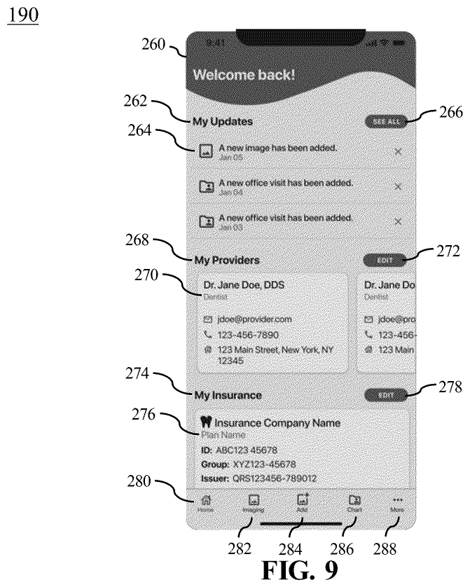

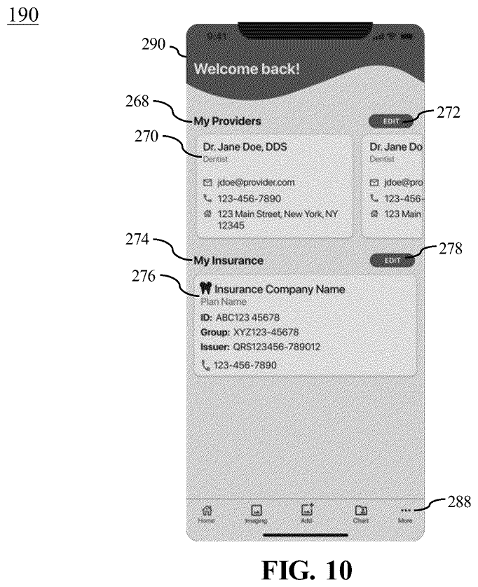

With reference to FIGS. 9 and 10, user interface 190 presents a patient with a dashboard view 260. Dashboard view 260 presents the patient with information relevant to their patient account. As an example, dashboard view 260 presents the patient with an updates section 262 that comprises information about the most recent updates 264 relating to their patient account. The patient may also select a see all element 266 to be presented with a list of all relevant updates for their patient account.

Dashboard view 260 also presents the patient with a my providers section 268 that provides information about the patient's associated dental providers. As an example, my providers section 268 may comprise a dental provider card 270 for each associated dental provider. The dental provider card 270 for each dental provider may comprise information such as, e.g., a name of the dental provider, contact information for the dental provider, an address of the dental provider or any other information about the dental provider that is stored in dental provider database 174. Dental provider card 270 is selectable by the patient to view additional information about the provider particular. The patient may also edit their list of dental providers by selecting an edit providers element 272.

Dashboard view 260 also presents the patient with a my insurance section 274 that provides information about the patient's associated dental insurance. As an example, my insurance section 274 may comprise an insurance card 276 for each associated dental insurance plan that the patient participates in. The insurance card 276 for each dental insurance plan may comprise information such as, e.g., a name of the dental insurance provider, an ID number for the dental insurance plan, a group number for the dental insurance plan, an issuer number for the dental insurance plane or any other information about the dental insurance plan that is stored in dental provider database 174. Insurance card 276 is selectable by the patient to view additional information about the dental insurance plan. The patient may also edit their dental insurance plan by selecting an edit insurance element 278.

Dashboard view 260 also presents the patient with a home element 280, imaging element 282, add element 284, chart element 286 and more element 288. The patient may select the home element 280 at any time to return to dashboard view 260. The patient may select imaging element 282 to view any images found in the EDR files associated with the patient account that are stored on EDR database 170. The patient may select add element 284 to initialize EDR capture module 140 for adding additional EDR files to EDR database 170 in association with the patient account. The patient may select chart element 286 to view a patient chart associated with the patient account. As an example, patient charts, treatment summaries or other patient information associated with dental provider activities may be stored in EDR database 170 and may be accessed by the patient by selecting chart element 286. The patient may select more element 288 to view a list of other available views. For the sake of brevity, elements 280-288 will only be labeled in FIG. 9 and where necessary to support a description of a particular view and will not be separately labeled on each figure of user interface 190. Similar functionality to elements 280-288 may be found on one or more other views of user interface 190 whether or not elements 280-288 are shown.

Dashboard view 260 may be a dynamic view where, for example, only relevant information may be presented to the patient. As an example, while my updates section 262 is shown in FIG. 9 with most recent updates 264, in a case where no recent updates 264 are present, my updates section 262 may be removed from presentation, e.g., as shown in FIG. 10. If the patient desires to view older updates, the patient may select more element 288 for a list including an option to view all updates that has similar functionality to see all element 266 as described above.

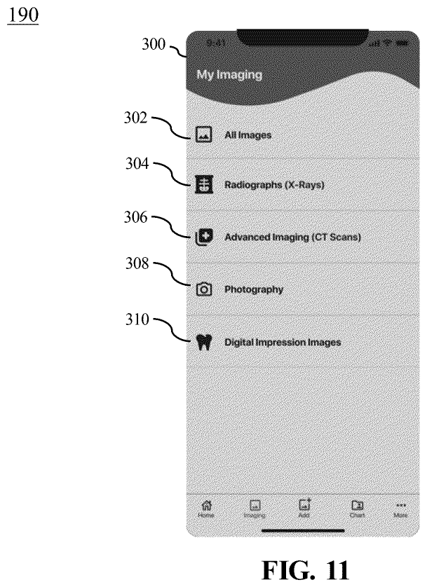

With reference to FIG. 11, a patient selection of imaging element 282 causes user interface 190 to present the patient with a view 300. View 300 presents the patient with elements that are selectable to view various images associated with their EDR files. As an example, view 300 presents the patient with an all images element 302, radiographs (x-rays) element 304, advanced imaging (CT Scans) element 306, photography element 308 and digital impressions images element 310. Any of elements 302-310 are selectable by the patient to view a corresponding listing of images or files of that type in user interface 190.

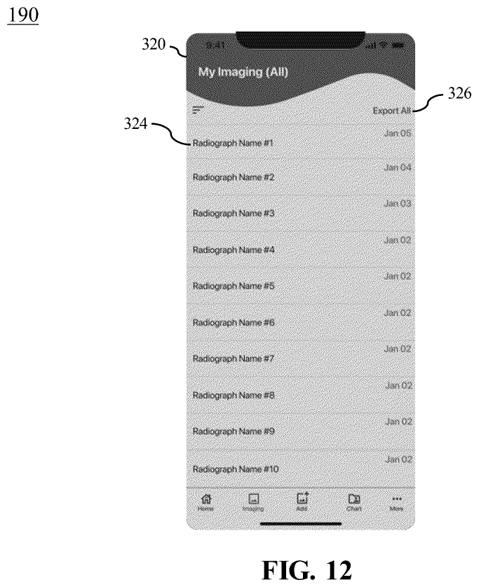

With reference to FIG. 12, a patient selection of one of elements 302-310 causes user interface 190 to presents the patient with a view 320 comprising a list 322 of EDR files 324 that may be selected for viewing by the patient. As an example, if the patient selected radiographs (x-rays) element 304 form view 300, user interface 190 may present the patient with view 320 comprising a list 322 of radiograph EDR files 324 that may be selected by the patient. A selection of a particular EDR file 324 from list 322 may present the patient with an image of the corresponding image or content of the EDR file 324. The patient may be given the option to export all EDR files 324 in the list 322 by selection of an export all element 326 to activate EDR export module 150.

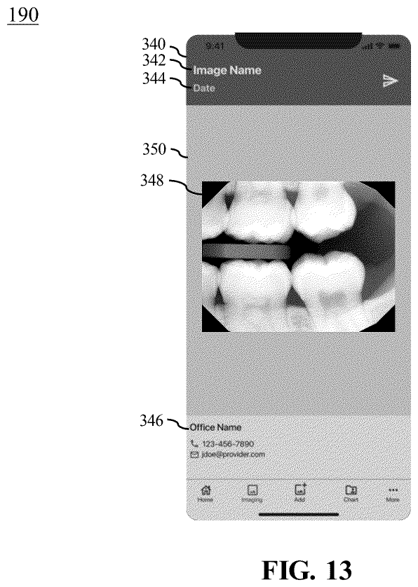

With reference to FIG. 13, a selection of a particular EDR file 324 from list 322 in FIG. 12 may cause user interface 190 to present the patient with a view 340. View 340 presents the patient with details associated with the selected EDR file 324 including, for example, a name 342 of the EDR file, a date 344 that the EDR file was generated and dental provider information 346 comprising a name and contact information for the dental provider. View 340 may also present a representation 348 of the content of the EDR file 224 in a content section 350, e.g., an image of the patient's teeth as shown in FIG. 13.

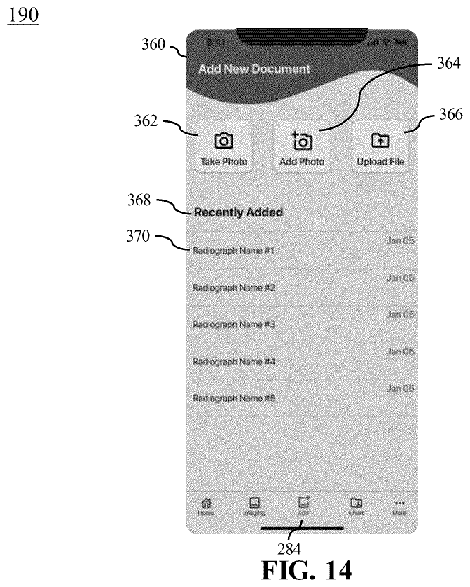

With reference to FIG. 14, a selection of add element 284 by a patient causes patient portal module 130 to activate EDR capture module 140. EDR capture module 140 causes user interface 190 to present the patient with a view 360. View 360 presents the patient with a take photo element 362, add photo element 364, upload file element 366 and a list 368 of recently added EDR files 370.

Take photo element 362 is selectable by the patient to activate scan capture module 146 (FIG. 3). For example, activation of take photo element 362 may cause scan capture module 146 to integrate with an image capture device of the patient's mobile device to capture image data, process the image data, and store the image data as a new EDR file associated with the patient in EDR database 170.

Add photo element 364 is selectable by the patient to active upload capture module 144 (FIG. 3). For example, activation of add photo element 364 may cause upload capture module 144 to present the user with a field for identifying a location of an image to be uploaded to EDR database 170 and for selecting the image to be uploaded. Upload capture module 144 uploads and stores the selected image as a new EDR file associated with the patient in EDR database 170.

Upload file element 366 is selectable by the patient to active upload capture module 144 (FIG. 3). For example, activation of upload file element 364 may cause upload capture module 144 to present the user with a field for identifying a location of a patient file to be uploaded to EDR database 170 and for selecting the patient file to be uploaded. Upload capture module 144 uploads and stores the selected upload file as a new EDR file associated with the patient in EDR database 170.

List 368 presents the patient with an indication of recently added EDR files 370 including information such as, e.g., a file name and date. In some embodiments, the patient may select a recently added EDR file 370 to view the contents of the file, e.g., images, text, or any other content.

In some embodiments, view 360 may also or alternatively present the patient with an activatable element to activate email capture module 142. Activate email capture module 142 may then parse through a corresponding email account to identify and capture dental images and other dental files associated with the patient. Email capture module 142 stores any images, files or other content associated with the patient's dental records as new EDR files associated with the patient in EDR database 170.

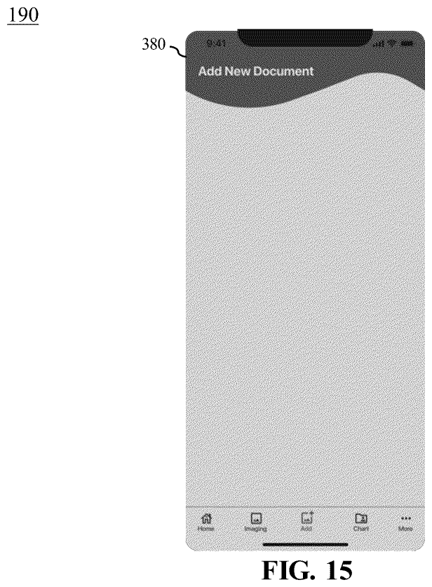

With reference to FIG. 15, user interface 190 presents the patient with a view 380 for adding new documents.

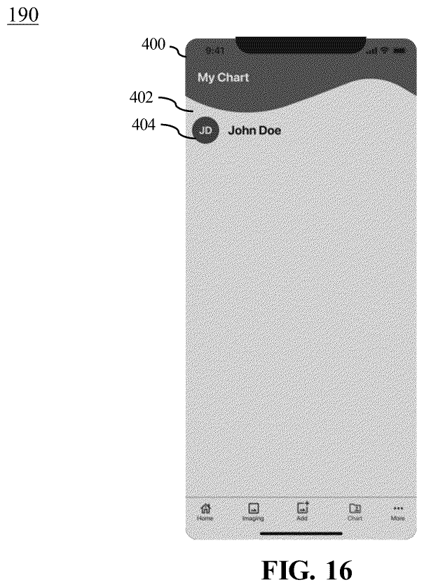

With reference to FIG. 16, a selection of chart element 286 by a patient causes user interface 190 to present the patient with a view 400 comprising a list 402 of available patient charts 404. As an example, if the patient is responsible for one or more minors, elders or other family members, each individual may have a separate patient chart 404 in the list 402. The patient may select a corresponding patient chart 404 to view the chart and other information for that individual, effectively switching which individual's dental information and details are presented by user interface 190. If the patient wishes to switch back to their own chart or view another chart, they may select a different patient chart 404.

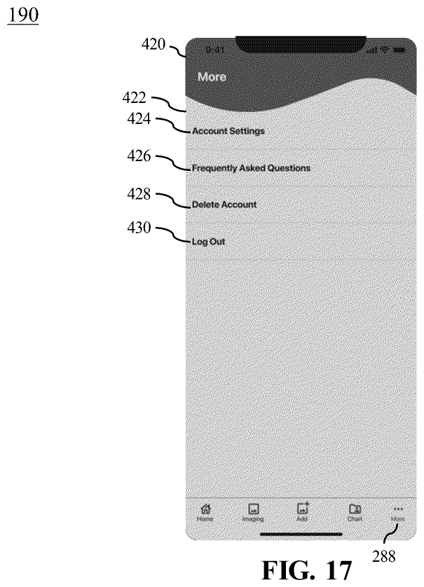

With reference to FIG. 17, a selection of more element 288 by a patient causes user interface 190 to present the patient with a view 420 comprising a list 422 of activatable elements that may be selected by the patient. As an example, list 422 may comprise an account settings activatable element 424 that is selectable by the patient to modify the information about the patients account, a frequently asked questions element 426 that is selectable by the patient to review a list of frequently asked questions, a delete account element 428 that is selectable by the patient to delete the patient's account and a log out element 430 that is selectable by the patient to cause account module 134 (FIG. 2) to log the patient out of system 100. In some embodiments, the patient may also be logged out by closing the application implemented by computing system 110 to present user interface 190 on computing device 180, e.g., a mobile application, web browser or any other application.

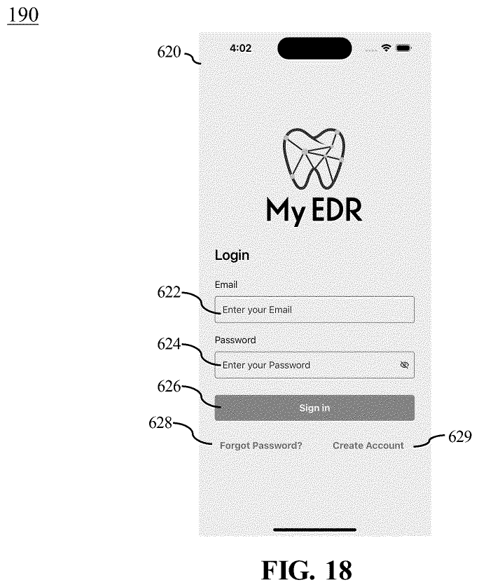

According to an embodiment, with reference to FIG. 18, user interface 190 presents the patient with view 620 to sign into their account. View 620 comprises an email field 622 and password field 624. View 620 further comprises a sign in element 626, a forgot password element 628, and a create account element 629. Account module 134 may receive login information from the patient, e.g., via the interactive patient portal, and compare the login information to account information found in patient database 172. In some embodiments, view 620 may also or alternatively provide the capability to establish other modes of authentication including, e.g., fingerprint scanning, retinal scanning, facial recognition, multi-factor authentication or any other manner of authentication.

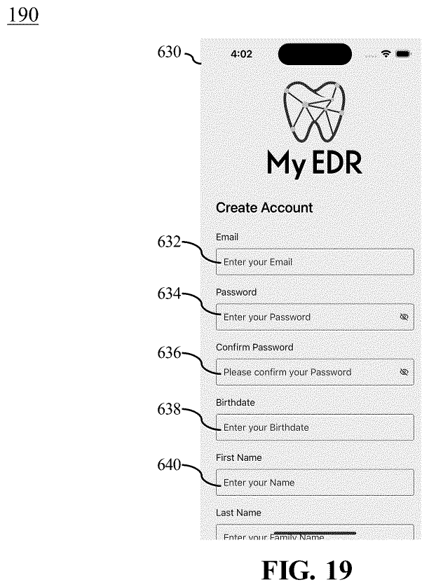

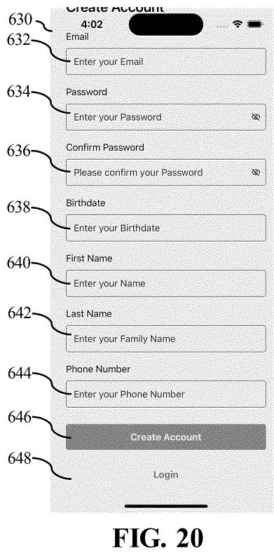

With reference to FIGS. 19 and 20, user interface 190 presents the patient with a view 630 to create an account. Upon selection of the create account element 629, the system 100 presents view 630. View 630 comprises an email field 632, password field 634, confirm password field 636, birthdate field 638, first name field 640, last name field 642, and phone number field 644. View 630 further comprises a create account element 646 and login element 648. The patient may, for example, enter their login credentials such as email, password, and the like in fields 632-644 and confirm their information by selecting create account element 646 to create the account. The patient account information is stored by account module 134 in patient database 172.

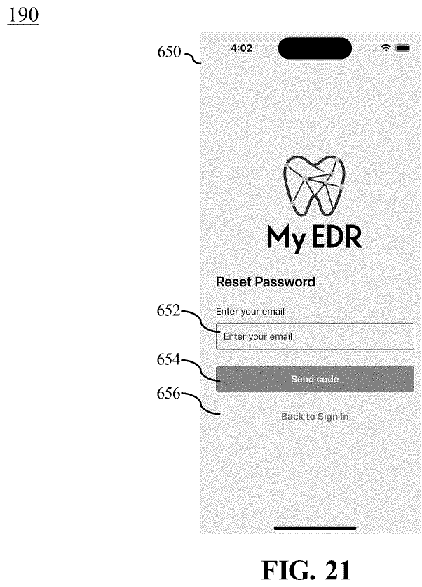

With reference to FIG. 21, user interface 190 presents the patient with a view 650. Upon selection of the forgot password element 628, the system 100 presents view 650. View 650 comprises an email field 652, a send code element 654, and a back to sign in element 656.

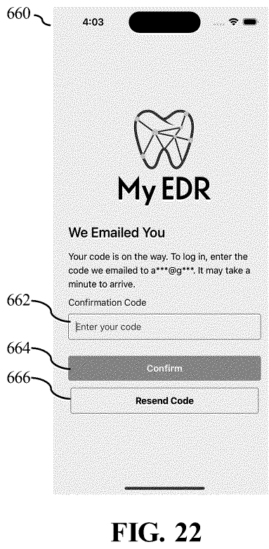

With reference to FIG. 22, user interface 190 presents the patient with a view 660. Upon selection of the send code element 654, the system 100 emails a confirmation code to the email address associated with the patient account. The confirmation code may be entered into confirmation code field 662. View 660 further comprises a confirm element 664 and a resend code element 666.

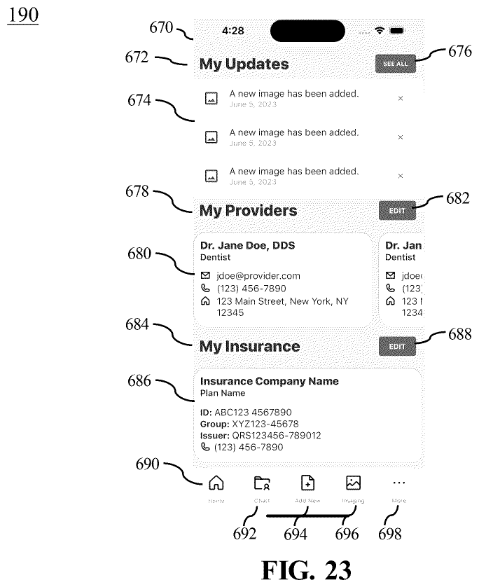

With reference to FIG. 23, user interface 190 presents the patient with a dashboard view 670. Dashboard view 670 presents the patient with information relevant to their patient account. As an example, dashboard view 670 presents the patient with an updates section 672 that comprises information about the most recent updates 674 relating to their patient account. The patient may also select a see all element 676 to be presented with a list of all relevant updates for their patient account.

Dashboard view 670 also presents the patient with a my providers section 678 that provides information about the patient's associated dental providers. As an example, my providers section 678 may comprise a dental provider card 680 for each associated dental provider. The dental provider card 680 for each dental provider may comprise information such as, e.g., a name of the dental provider, contact information for the dental provider, an address of the dental provider or any other information about the dental provider that is stored in dental provider database 174. Dental provider card 680 is selectable by the patient to view additional information about the provider. The patient may also edit their list of dental providers by selecting an edit providers element 682.

Dashboard view 670 also presents the patient with a my insurance section 684 that provides information about the patient's associated dental insurance. As an example, my insurance section 684 may comprise an insurance card 686 for each associated dental insurance plan that the patient participates in. The insurance card 686 for each dental insurance plan may comprise information such as, e.g., a name of the dental insurance provider, an ID number for the dental insurance plan, a group number for the dental insurance plan, an issuer number for the dental insurance plane or any other information about the dental insurance plan that is stored in dental insurance database 176. Dental insurance database 176 may be populated by dental insurance information provided by patients, dental providers, directly from dental insurance providers or in any other manner. Insurance card 686 is selectable by the patient to view additional information about the dental insurance plan. The patient may also edit their dental insurance plan by selecting an edit insurance element 688.

Dashboard view 670 also presents the patient with a home element 690, chart element 692, add element 694, imaging element 696 and more element 698. The patient may select the home element 690 at any time to return to dashboard view 670. The patient may select chart element 692 to view a patient chart associated with the patient account. The patient may select add element 694 to initialize EDR capture module 140 for adding additional EDR files to EDR database 170 in association with the patient account. The patient may select imaging element 696 to view any images found in the EDR files associated with the patient account that are stored on EDR database 170. As an example, patient charts, treatment summaries or other patient information associated with dental provider activities may be stored in EDR database 170 and may be accessed by the patient by selecting chart element 692. The patient may select more element 698 to view a list of other available views. For the sake of brevity, elements 690-698 will only be labeled in FIG. 23 and where necessary to support a description of a particular view and will not be separately labeled on each figure of user interface 190. Similar functionality to elements 690-698 may be found on one or more other views of user interface 190 whether or not elements 690-698 are shown.

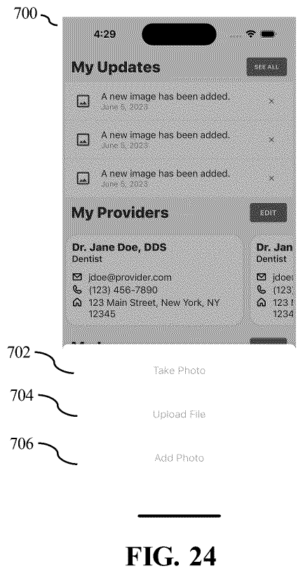

With reference to FIG. 24, user interface 190 presents the patient with view 700. Selection of the add element 694 causes patient portal module 130 to activate EDR capture module 140. EDR capture module 140 causes user interface 190 to present the patient with view 700. View 700 presents the patient with a take photo element 702, upload file element 704, and add photo element 706.

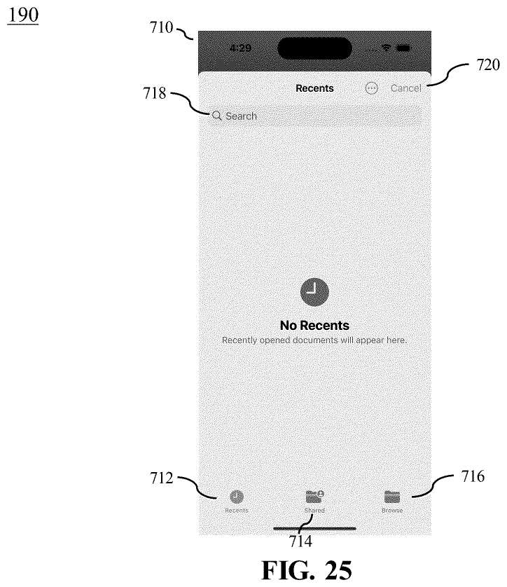

With reference to FIG. 25, user interface 190 presents the patient with view 710. Upon selection of the upload file element 704, the system 100 presents view 710 from which the patient may select and add files to EDR database 170 in association with the patient account. Upload capture module 144 is configured to obtain files provided by the patient via an upload mechanism. Such files may be uploaded directly from, for example, the patient's mobile device or a cloud account. View 710 comprises a recents element 712, shared element 714, and browse element 716. View 710 further comprises a search element 718 and a cancel element 720.

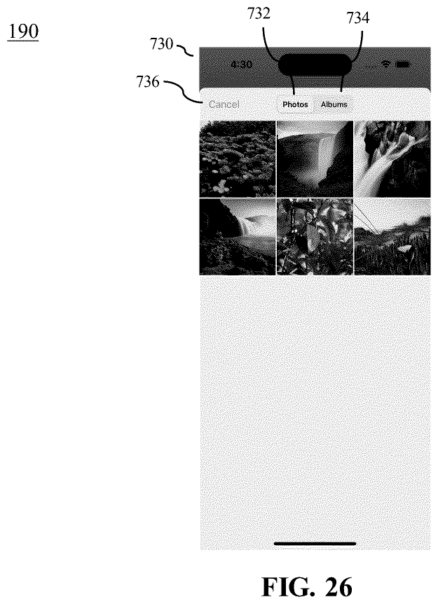

With reference to FIG. 26, user interface 190 presents the patient with view 730. Upon selection of the add photo element 706, the system 100 presents view 730 from which the patient may select and add photos to EDR database 170 in association with the patient account. Such photos may be uploaded directly from, for example, the patient's mobile device or a cloud account. View 730 comprises a photos element 732, an albums element 734, and a cancel element 736.

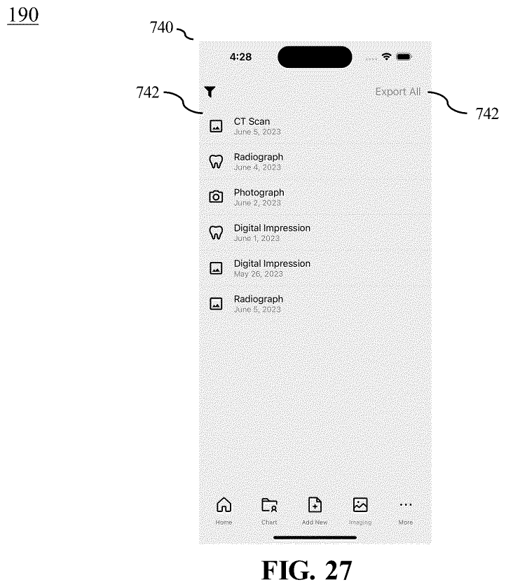

With reference to FIG. 27, a patient selection of imaging element 696 causes user interface 190 to present the patient with a view 740. View 740 presents the patient with elements that are selectable to view various images associated with their EDR files. View 740 presents the patient with a list 742 of images or files, such as advanced imaging or CT scans, radiographs (x-rays), digital impression images, and photographs associated with their EDR files. The list 742 may be organized, for example, by date, type, name, file size, or by custom order. Any of the images or files in list 742 are selectable by the patient to view in user interface 190. View 740 further comprises an export all element 744 configured to allow the patient to export all EDR files in the list 742, e.g., via EDR export module 150.

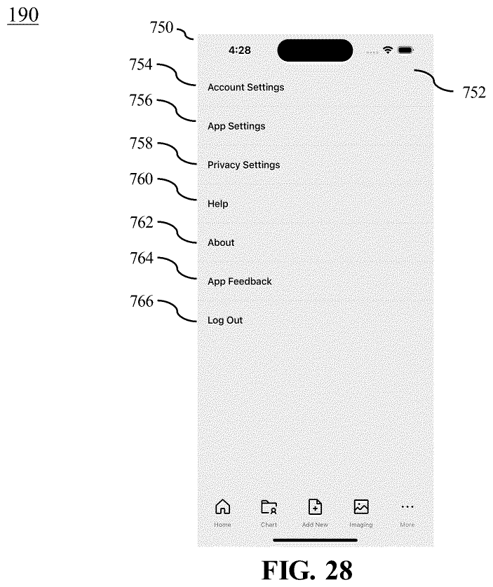

With reference to FIG. 28, a selection of more element 698 by a patient causes user interface 190 to present the patient with a view 750 comprising a list 752 of activatable elements that may be selected by the patient. As an example, list 752 may comprise an account settings activatable element 754 that is selectable by the patient to modify the information about the patient's account, an app settings element 756 that is selectable by the patient to view and configure current application settings, a privacy settings element 758 that is selectable by the patient to view and configure current privacy settings, a help element 760 that is selectable by the patient to view, for example, frequently asked questions and receive customer support, an about element 762 that is selectable by the patient to view detailed information pertaining to the application, an app feedback element 764 that is selectable by the patient to submit feedback on the application, and a log out element 766 that is selectable by the patient to cause account module 134 (FIG. 2) to log the patient out of system 100. In some embodiments, the patient may also be logged out by closing the application implemented by computing system 110 to present user interface 190 on computing device 180, e.g., a mobile application, web browser or any other application.

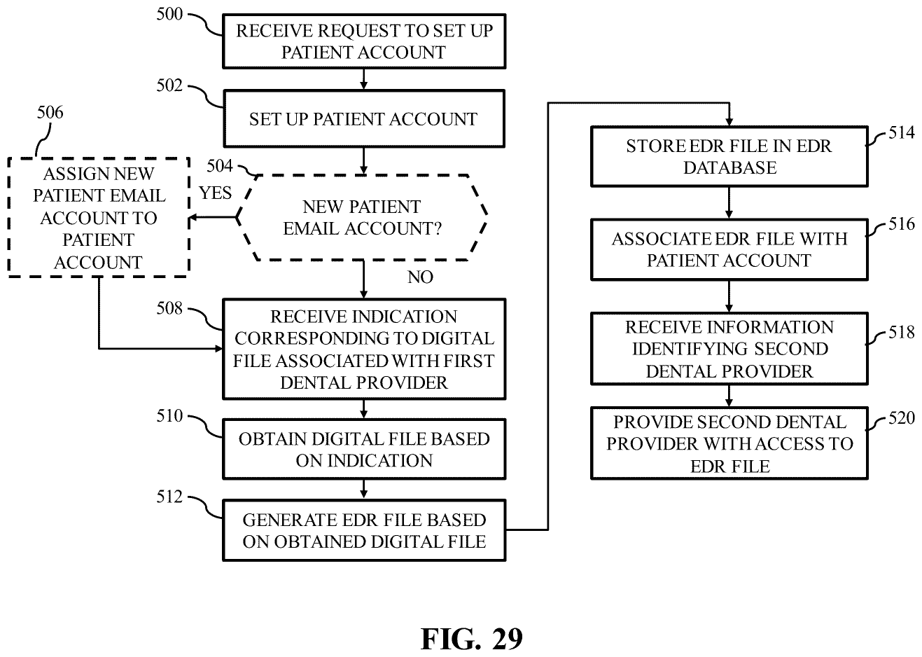

With reference to FIG. 29, a process of using system 100 to store, manage and maintain patient EDR files will now be described. The process of FIG. 29 comprises steps 500 through 520 and is suitable for use in the system 100 but is more generally applicable to other types of systems for storing, managing and maintaining patient EDR files.

At step 500, computing system 110 receives a request to set up patient account from computing device 180, e.g., via user interface 190.

At step 502, account module 134 sets up the patient account, for example, as described above.

At step 504, in some embodiments, account module 134 may optionally determine whether or not the patient account requires a new patient email account. As an example, the patient may select an element in user interface 190 that indicates to account module 134 that the patient desires to use an email account provided by system 100. If account module 134 determines that the patient account requires a new patient email account, the process proceeds to step 506. If account module 134 determines that the patient account does not require a new patient email account, the process proceeds to step 508.

At step 506, account module 134 assigns a new patient email account to the patient account. Account module 134 may provide details of the new patient email account to the patient, e.g., via user interface 190. The process then proceeds to step 508.

Steps 504 and 506 may be optional and provided in some embodiments but not in other embodiments as desired.

At step 508, computing system 110 receives an indication corresponding to digital file associated with first dental provider, e.g., from the patient via user interface 190 of computing device 180.

At step 510, EDR capture module 140 obtains the digital file based on indication. As an example, email capture module 142, upload capture module 144 or scan capture module 146 may be utilized to obtain the digital file. In some embodiments, for example, the module 142, 144 and 146 utilized to obtain the digital file may depend on the type of the indication or information associated with the indication, e.g., if the indication is that an email having the digital file was received, email capture module 142 may be utilized, if the indication is of a location where the digital file can be found, upload capture module 144 may be utilized, if the indication is that an image capture device has been activated, scan capture module 146 may be utilized, or any other indication or information may be utilized.

At step 512, EDR capture module 140 generates an EDR file based on obtained digital file.

At step 514, EDR capture module 140 stores the generated EDR file in EDR database 170.

At step 516, account module 134 associates the EDR file with the patient account, e.g., by creating a link between the patient account and the EDR file stored in EDR database 170.

At step 518, computing device 110 receives information identifying a second dental provider, e.g., from the patient via user interface 190 of computing device 180.

At step 520, computing device 110 provides the second dental provider with access to the EDR file. As an example, EDR export module 150 may be utilized to provide the second dental provider with access to the EDR file as described above.

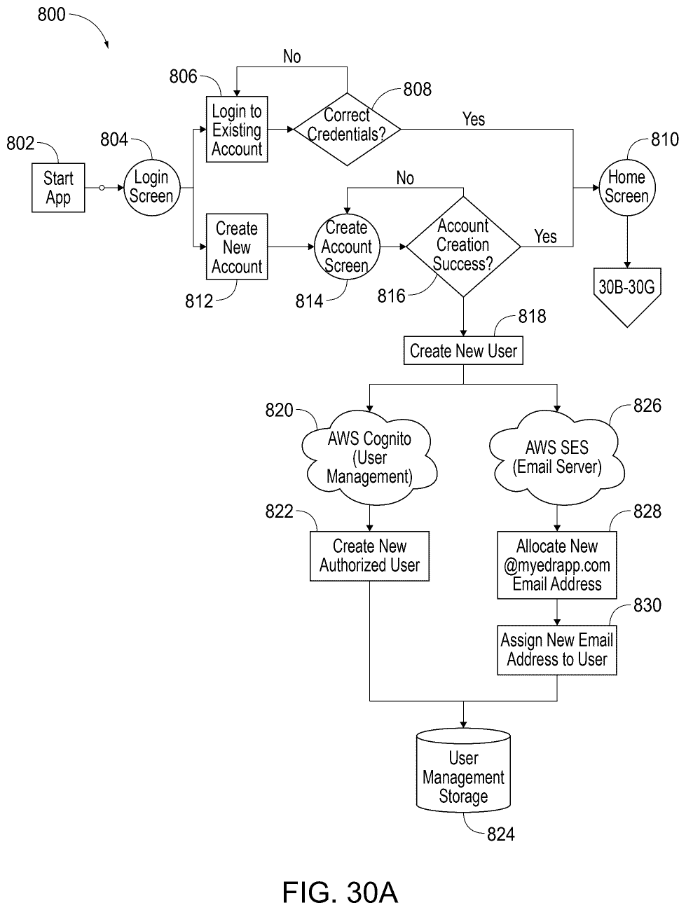

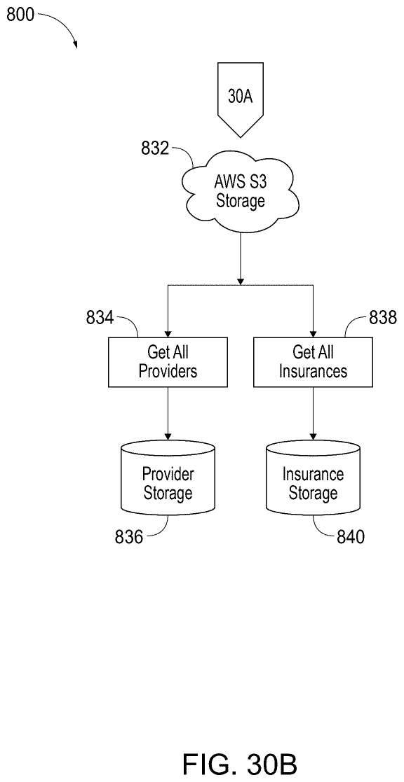

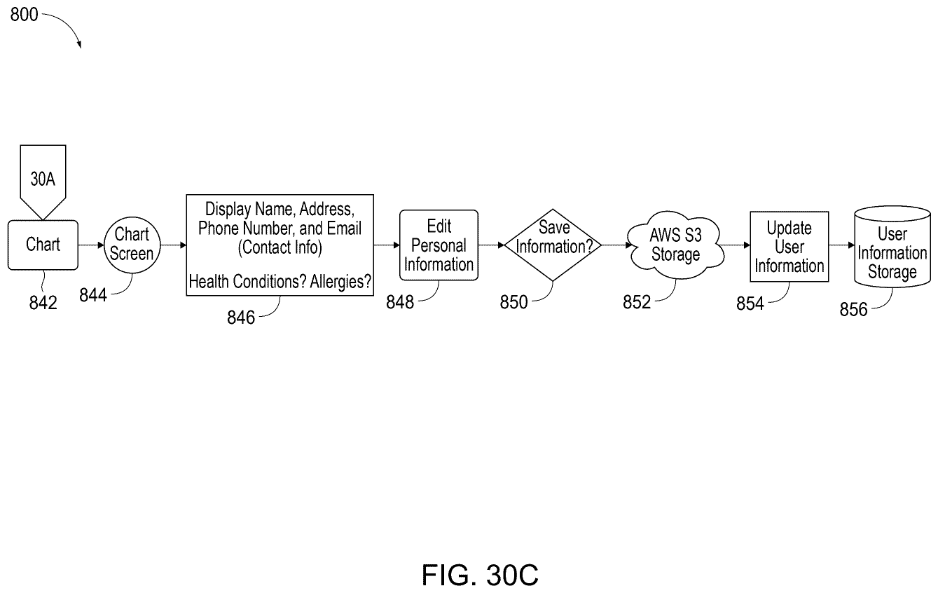

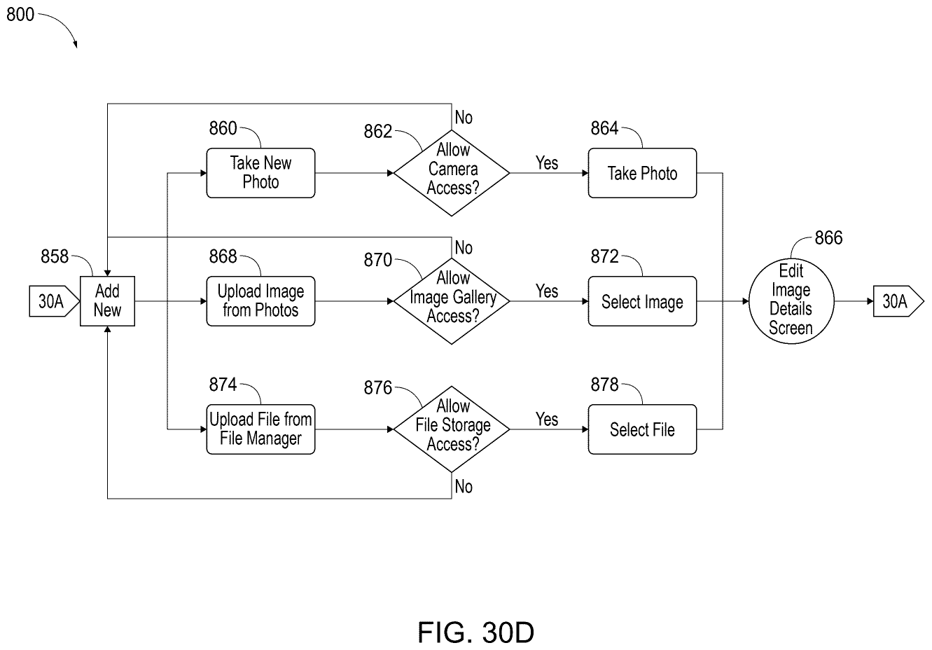

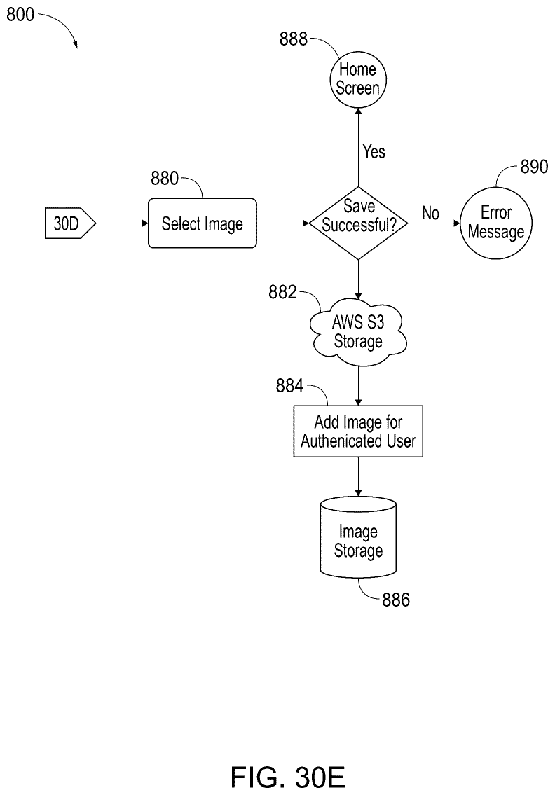

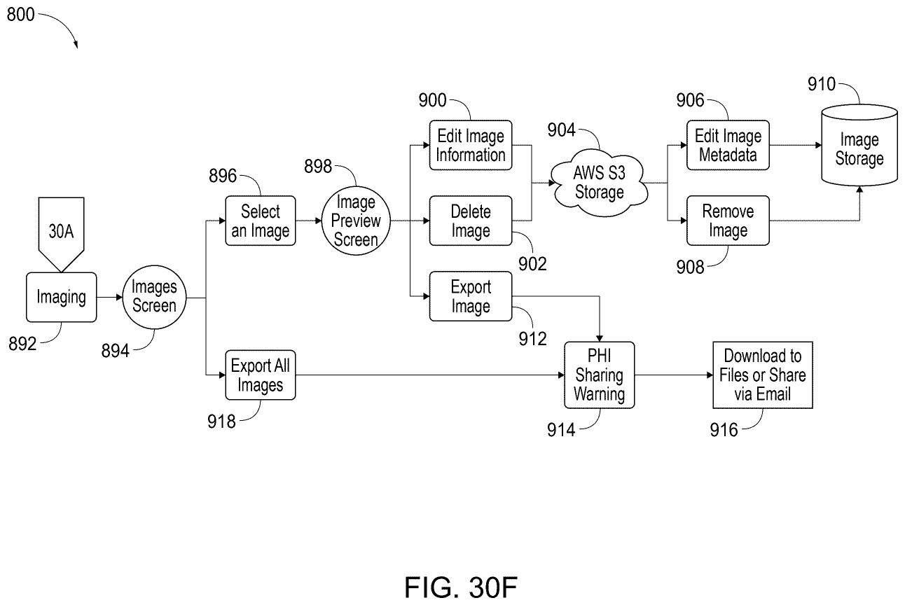

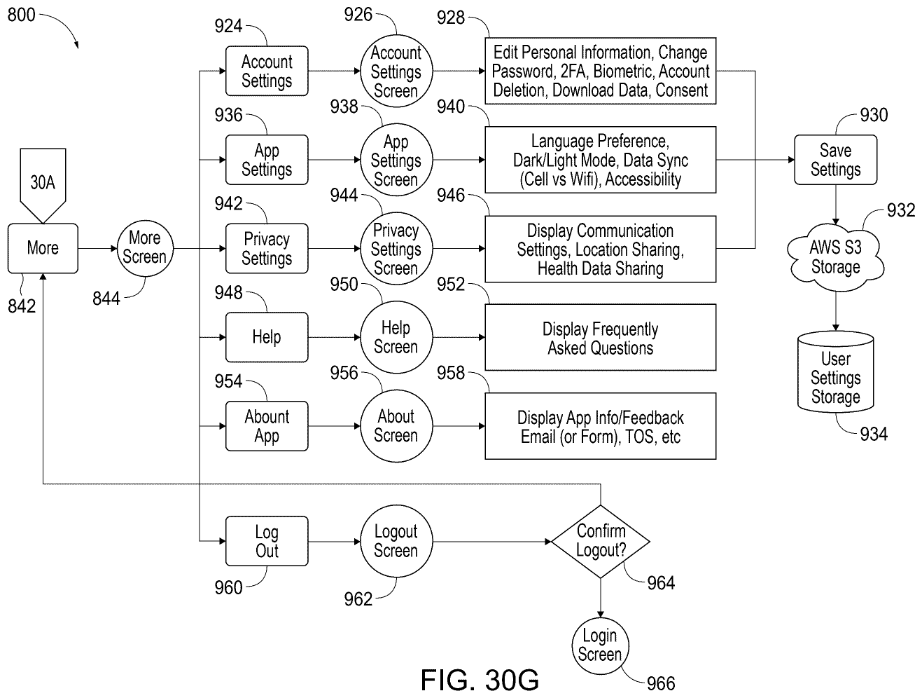

With reference to FIG. 30, an overall process 800 of using system 100 will now be described. With reference to FIG. 30A, upon starting the application implemented by computing system 110 (step 802), the patient is presented with a login screen (step 804, view 620) to sign into an existing account or to create a new account. The patient may login to existing account (step 806, by selecting the sign in element 626 and entering their credentials). Alternatively, the patient may create a new account (steps 812 and 814, by selecting the create account element 629, entering their information in view 630, and selecting the create account element 646). The new account is created (step 818) using a cloud user management (step 820), for example, AWS Cognito, which is configured to create a new authorized user (step 822) for storage in, for example, patient database 172 (step 824). A cloud email server (step 826), for example, AWS SES, is configured to allocate and assign a new email address to the patient (steps 828 and 830) for storage in, for example, patient database 172 (step 824). Upon validation of the patient's login credentials (step 808) or creation of a new account (step 816), the system 100 presents a home screen (step 810, dashboard view 670).

With reference to FIG. 30B, the home screen presents the patient with information about the patient's associated dental providers (in the my providers section 678, step 834) and information about the patient's associated dental insurance (in the my insurance section 684, step 838) using a cloud service (step 832), for example AWS S3 storage. The provider and insurance information are stored in the dental provider database 174 (step 836) and dental insurance database 176 (step 840), respectively.

As discussed above, the home screen presents the patient with chart element 692, an add element 694, imaging element 696 and more element 698. With reference to FIG. 30C, the patient may select chart element 692 (step 842) to view a chart screen (step 844) displaying a patient chart associated with the patient account. The patient's name, address, phone number, email, and other contact information, as well as health conditions and allergies are displayed (step 846). The patient may select an edit personal information element to edit (step 848) and save (step 850) their personal information. Using a cloud service (step 852) such as AWS S3 storage, the updated personal information (step 854) is stored in the patient database 172, while any medical information may be stored in the EDR database 170 (step 856).

With reference to FIG. 30D, the patient may select add element 694 (step 858) to initialize EDR capture module 140 for adding additional EDR files to EDR database 170 in association with the patient account. The patient may take a new photo (take photo element 702, step 860), upload an image from photos (add photo element 706, step 868), and upload a file from a file manager (upload file element 704, step 874). To take a new photo, the system 100 will prompt the patient to allow camera access (step 862). Once access is granted, the patient may take a photo (step 864) and edit image details (step 866). To upload an image from photos, the system 100 will prompt the patient to allow image gallery access (step 870). Once access is granted, the patient may select an image (step 872) stored on their device or on a cloud account and edit image details (step 866). To upload a file from the file manager, the system 100 will prompt the patient to allow file storage access (step 876). Once access is granted, the patient may select a file (step 878) stored on their device or on a cloud account and edit image details (step 866).

With reference to FIG. 30E, the patient may select the new image (step 880). Using a cloud service (step 882) such as AWS S3 storage, the patient may save the new image for an authenticated user (step 884) for storage (step 886) in the EDR database 170. A successful attempt to save the new image will result in the system 100 displaying the home screen (step 888). An unsuccessful attempt to save the new image will result in the system 100 displaying an error message screen (step 890).

With reference to FIG. 30F, the patient may select imaging element 696 (step 892) to view any images found in the EDR files associated with the patient account that are stored on EDR database 170. An images screen (view 740, step 894) presents the patient with elements that are selectable to view various images associated with their EDR files. The patient may select (step 896) any of the images or files presented on the images screen to view in an image preview screen (step 898). The patient may select an edit image information element (step 900) or a delete image element (step 902). Using a cloud service (step 904) such as AWS S3 storage, any edits to the image metadata (step 906) or image deletions (step 908) are updated for storage (step 910) on EDR database 170. The patient may also select an export image element (step 912). Before the selected image is downloaded to the patient's files or shared via email, the system 100 will issue a personal health information (PHI) sharing warning (step 914). The patient may acknowledge the warning and proceed with the image export (step 916). The images screen further comprises an export all element 744 (step 918) which the patient may select to export all EDR files, e.g., via EDR export module 150. As above, the system 100 will issue a PHI sharing warning (step 914) before downloading the images to the patient's files or sharing the images via email (step 916).

With reference to FIG. 30G, the patient may select more element 698 (step 920) to view a more screen (view 750, step 922)) comprising a list 752 of activatable elements that may be selected by the patient. The patient may select account settings (account settings activatable element 754, step 924) to view an account settings screen (step 926) to edit their personal information, change their password, enable or disable two-factor or biometric authentication, delete their account, edit settings related to downloading data, and edit their consent data (step 928). The patient may select app settings (app settings element 756, step 936) to view an app settings screen (step 938) to update their language preferences, switch between dark and light screen modes, configure data sync preferences, e.g., cellular data or Wi-Fi, and configure accessibility functionality (step 940). The patient may select privacy settings (privacy settings element 758, step 942) to view a privacy settings screen (step 944) to display and configure settings related to communication, location sharing, and health data sharing (step 946). Any changes to account settings, app settings, and privacy settings may be saved (step 930) using a cloud service (step 932) such as AWS S3 storage for storage in a user settings storage database or patient database 172 (step 934).

The more screen further comprises a help element (760) which a patient may select (step 948) to view a help screen (step 950) displaying frequently asked questions (step 952), and an about element (762) which a patient may select (step 954) to view an about screen (step 956) displaying app information, an option to submit feedback on the application via email or a form, and terms of service (step 958). The more screen further comprises a log out element (766) that is selectable by the patient (step 960) to view a logout screen (step 962) and confirm a logout (step 964). Once successfully logged out, the system 100 presents the login screen (step 966).

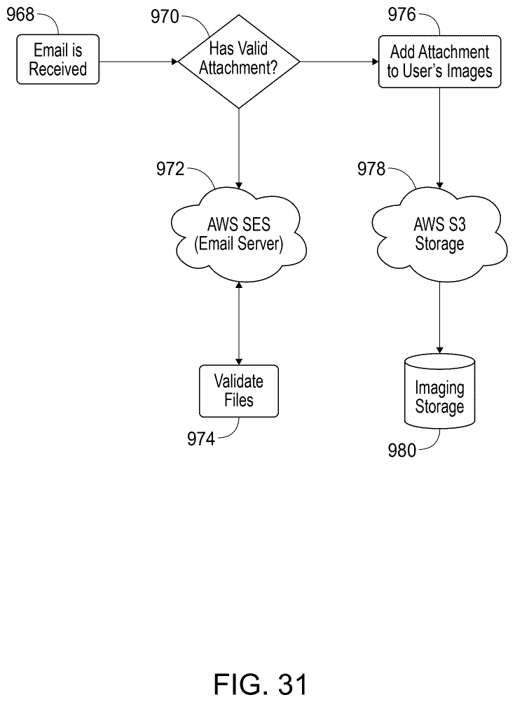

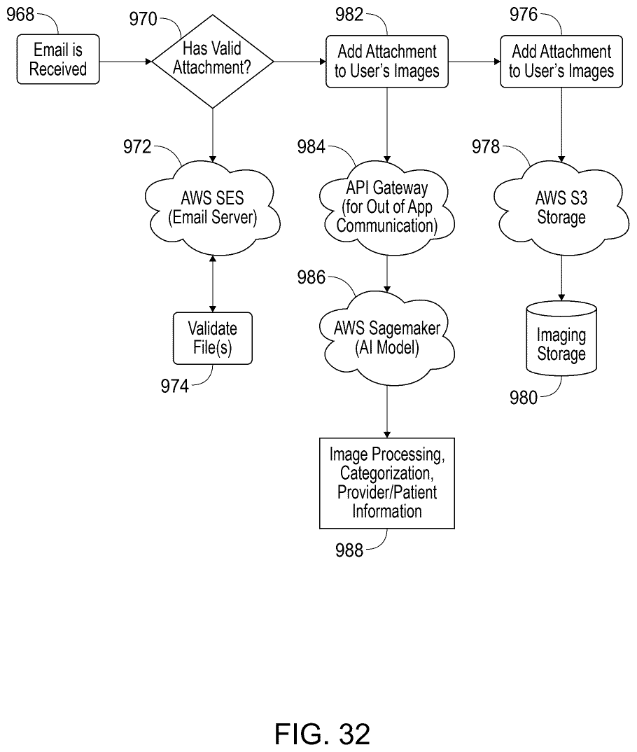

With reference to FIGS. 31 and 32, the system 100 may be used for an email integration process. The email integration process comprises receiving an email (step 968) and determining whether the email includes a valid attachment (step 970). For example, the patient may have received EDR files from a dental provider on their own email address or on the patient email address provided by system 100. Using a cloud email server, for example, AWS SES (step 972), file attachments may be validated (step 974). If the email includes a valid attachment, the attachment is added to the EDR database 170 in association with the patient account (step 976). The email integration process may utilize email capture module 142, which is configured to monitor and parse through the emails and email attachments, identify EDR files, and, using a cloud email server, for example, AWS SES (step 978), download any identified EDR files for storage in image storage or the EDR database 170 (step 980).

With reference to FIG. 32, the system 100 may be used for an email import process supported by artificial intelligence (AI). The email import process comprises the email integration process described above, with added AI functionality (step 982). A cloud-based API, such as API Gateway (step 984), may be used to interface with an AI model, such as AWS SageMaker (step 986). The API model is configured to parse through the email attachments and process and categorize files, images, and information such as patient and provider information (step 988). The parsed attachments are added to the EDR database 170 in association with the patient account.

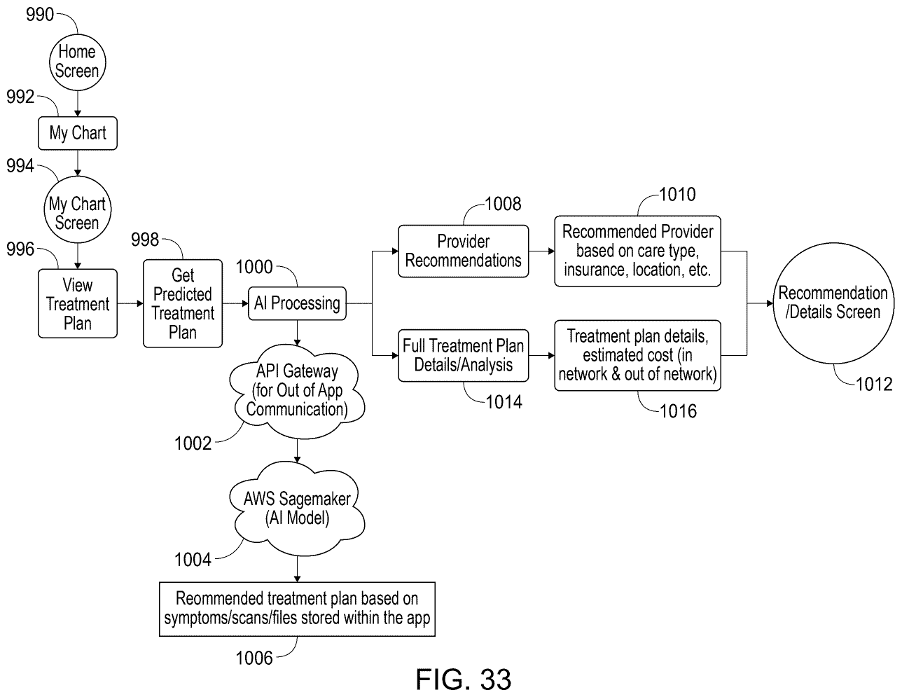

With reference to FIG. 33, the system 100 may be used for a treatment plan process supported by AI. From a home screen or dashboard view 260, 670 (step 990), the patient may select chart element 286, 692 (step 992) to view the patient chart associated with the patient account (step 994). The patient may select a view treatment plan element (step 996) and a get predicted treatment plan element (step 998). AI processing (step 1000) using a cloud-based API, such as API Gateway (step 1002), may be used to interface with an AI model, such as AWS SageMaker (step 1004). The API model is configured to recommend a treatment plan, including provider recommendations, based on the patient's symptoms, scans, and files stored within the EDR database 170 in association with the patient account (step 1006). The patient may select a provider recommendations element (step 1008) to view at least one recommended provider on a recommendation details screen (step 1012). The at least one recommended provider may be determined based on care type, insurance, and location (step 1010). The patient may also select a full treatment plan details and analysis element (step 1014) to view details pertaining to the treatment plan on the recommendation details screen (step 1012). Details of the treatment plan may include, for example, estimated costs for both in network and out of network providers (step 1016).

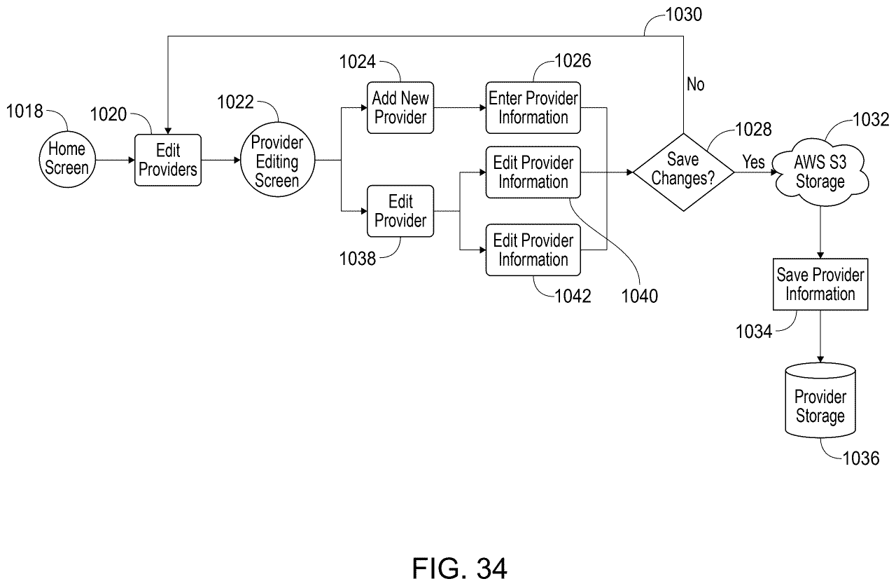

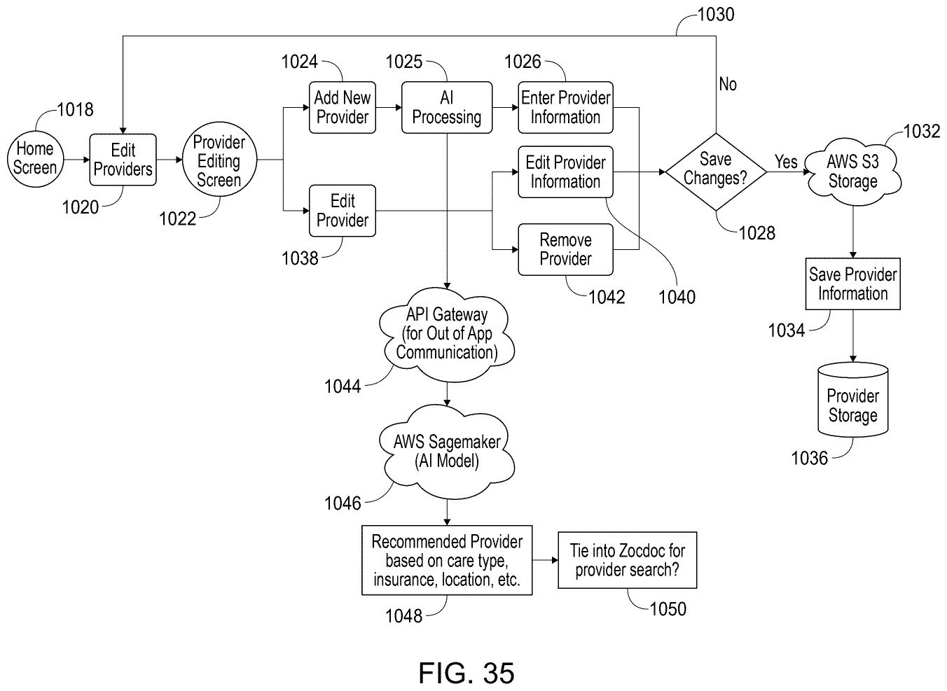

With reference to FIGS. 34 and 35, the system 100 may be used for an add provider process. From the home screen or dashboard view 260, 670 (step 1018), the patient may select the edit providers element 272, 682 (step 1020). Upon selection of the edit providers element, the system 100 presents a provider editing view (step 1022). The patient may select an add new provider element (step 1024) to enter provider information (step 1026). The patient may select an edit provider element (step 1038) to edit pre-existing provider information within the dental provider database 174 (step 1040) or to remove a pre-existing provider (step 1042). Once finished, the patient may save their changes (step 1028) or return to the provider editing view to make further changes (step 1030). Using a cloud service, for example AWS S3 storage (step 1032), any changes are saved (step 1034) to the dental provider database 174 (step 1036).

With reference to FIG. 35, the system 100 may be used for a provider search process supported by AI (step 1025). The provider search process comprises the add provider process described above, with added AI functionality. Upon selection of the add new provider element, a cloud-based API, such as API Gateway (step 1044), may be used to interface with an AI model, such as AWS SageMaker (step 1046). The API model is configured to recommend a provider based on care type, insurance, and location (step 1048). In an embodiment, the AI model is configured to search for a provider using information from a public online database such as Zocdoc® (step 1050).

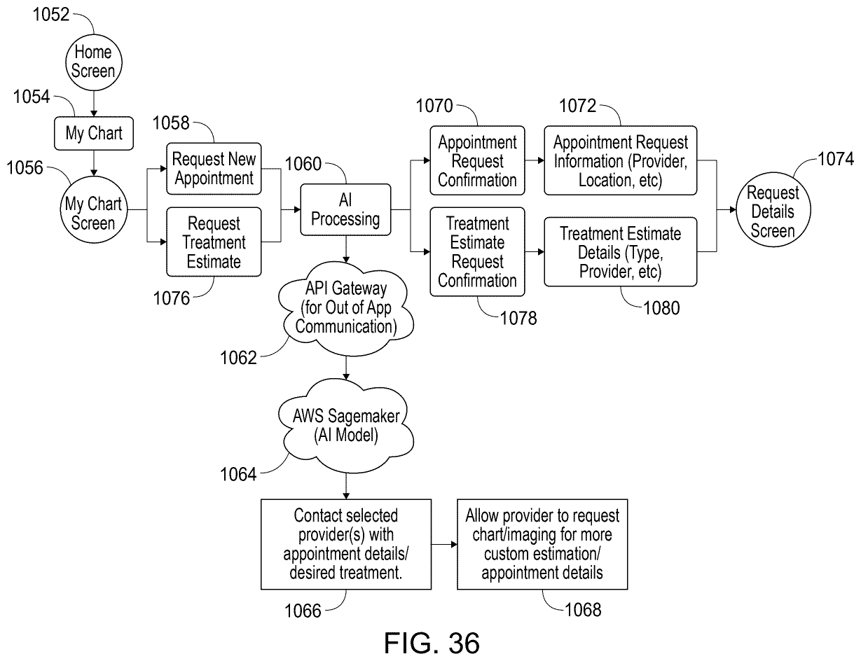

With reference to FIG. 36, the system 100 may be used for an appointment and estimate request process supported by AI. From the home screen or dashboard view 260, 670 (step 1052), the patient may select chart element 286, 692 (step 1054) to view the patient chart associated with the patient account (step 1056). The patient may select a request new appointment element (step 1058) or a request treatment estimate element (step 1076). Using AI processing (step 1060), upon selection of the request new appointment element or the request treatment estimate element, a cloud-based API, such as API Gateway (step 1062), may be used to interface with an AI model, such as AWS SageMaker (step 1064). The API model is configured to contact a patient-selected provider with appointment details, including desired treatment (step 1066). The API model is further configured to allow the selected provider to request the patient chart, including imaging associated with the patient account, in order to provide customized appointment and treatment estimate details (step 1068). The patient may select an appointment request confirmation element (step 1070) to view appointment request information, including the provider, location, and time (step 1072), on a request details view (step 1074). The patient may select a treatment estimate request confirmation (step 1078) to view treatment estimate details, including type of treatment, the provider, and costs (step 1080), on the request details view (step 1074). In an embodiment, the AI model is configured to schedule an appointment with a provider using a public online database such as Zocdoc®.

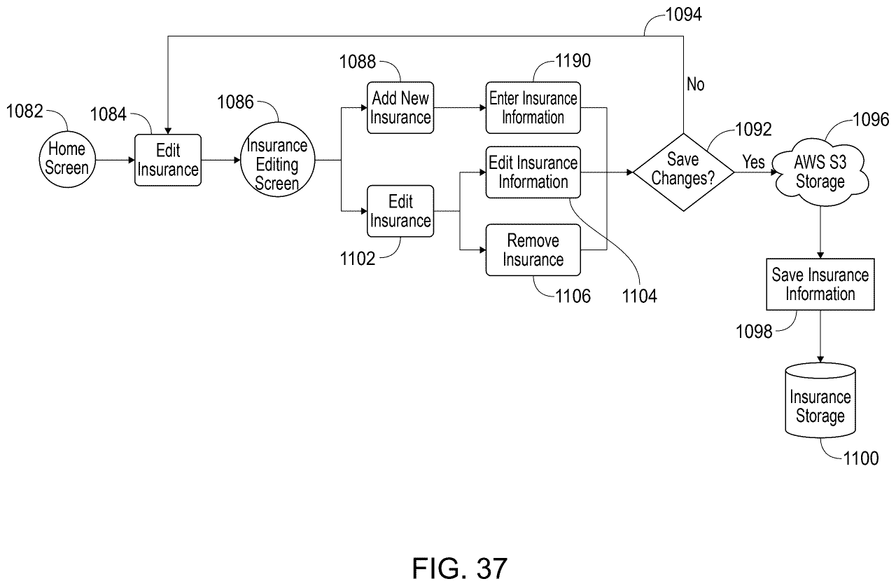

With reference to FIG. 37, the system 100 may be used for an add insurance process. From the home screen or dashboard view 260, 670 (step 1082), the patient may select the edit insurance element 278, 688 (step 1084) to view an insurance editing screen (step 1086). The patient may select an add new insurance element (step 1088) to enter information about their dental insurance plan (step 1090). The patient may select an edit insurance element (step 1102) to edit information about their dental insurance plan that is stored in dental insurance database 176 (step 1104), or to remove their dental insurance plan (step 1106). Once finished, the patient may save their changes (step 1092) or return to the provider editing view to make further changes (step 1094). Any changes are saved (step 1098) using a cloud service, for example AWS S3 storage (step 1096), to the dental insurance database 176 (step 1100).

The particular processing operations and other system functionality described in conjunction with the flow diagrams of FIG. 29-37 are presented by way of illustrative example only and should not be construed as limiting the scope of the disclosure in any way. Alternative embodiments can use other types of processing operations. For example, the ordering of the process steps may be varied in other embodiments, or certain steps may be performed at least in part concurrently with one another rather than serially. Also, one or more of the process steps may be repeated periodically, or multiple instances of the process can be performed in parallel with one another in order to implement the disclosed embodiments.

Functionality such as that described in conjunction with the processes of FIGS. 29-37 may be implemented at least in part in the form of one or more software programs stored in memory and executed by a processor of a processing device such as a computer or server. As will be described herein, a memory or other storage device having executable program code of one or more software programs embodied therein is an example of what is more generally referred to herein as a “processor-readable storage medium.”

FIGS. 1 through 37 are conceptual illustrations allowing for an explanation of the disclosed embodiments of the invention. Notably, the figures and examples above are not meant to limit the scope of the invention to a single embodiment, as other embodiments are possible by way of interchange of some or all of the described or illustrated elements. Moreover, where certain elements of the disclosed embodiments can be partially or fully implemented using known components, only those portions of such known components that are necessary for an understanding of the disclosed embodiments are described, and detailed descriptions of other portions of such known components are omitted so as not to obscure the disclosed embodiments. In the present specification, an embodiment showing a singular component should not necessarily be limited to other embodiments including a plurality of the same component, and vice-versa, unless explicitly stated otherwise herein. Moreover, terms in the specification or claims are not intended to be ascribed an uncommon or special meaning unless explicitly set forth as such. Further, the disclosed embodiments encompass present and future known equivalents to the known components referred to herein by way of illustration.

It should be understood that the various aspects of the embodiments could be implemented in hardware, firmware, software, or combinations thereof. In such embodiments, the various components and/or steps would be implemented in hardware, firmware, and/or software to perform the functions of the disclosed embodiments. That is, the same piece or different pieces of hardware, firmware, or module of software could perform one or more of the illustrated blocks (e.g., components or steps). In software implementations, computer software (e.g., programs or other instructions) and/or data is stored on a machine-readable medium as part of a computer program product and is loaded into a computer system or other device or machine via a removable storage drive, hard drive, or communications interface. Computer programs (also called computer control logic or computer-readable program code) are stored in a main and/or secondary memory, and executed by one or more processors (controllers, or the like) to cause the one or more processors to perform the functions of the invention as described herein. In this document, the terms “machine readable medium,” “computer-readable medium,” “computer program medium,” and “computer usable medium” are used to generally refer to media such as a random access memory (RAM); a read only memory (ROM); a removable storage unit (e.g., a magnetic or optical disc, flash memory device, or the like); a hard disk; or the like.

The foregoing description will so fully reveal the general nature of the disclosed embodiments that others can, by applying knowledge within the skill of the relevant art(s) (including the contents of the documents cited and incorporated by reference herein), readily modify and/or adapt for various applications such specific embodiments, without undue experimentation, without departing from the general concept of the disclosed embodiments. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

1. A system comprising at least one processor coupled to memory, the at least one processor being configured to:
maintain, by an account module, a patient account associated with a dental patient;
generate, by a user interface module, a user interface to be presented on a patient device associated with the dental patient;
receive, by a computing system, from the patient device, an indication corresponding to a digital file associated with the dental patient that was generated by a first dental provider;
obtain, by a capture module, based on the received indication, the digital file;
process and categorize the digital file using artificial intelligence (AI) and pattern matching;
generate, by the capture module, an electronic dental record (EDR) file based at least in part on the processed digital file;
store, by the capture module, the EDR file in an EDR database;
associate, by the account module, the EDR file with the patient account of the dental patient;
recommend, by a recommendation module, a patient treatment plan based on the EDR file, the patient treatment plan comprising at least a list of recommended dental providers and treatments;
receive, from the patient device, information identifying a second dental provider from the list of recommended dental providers;
provide, by an export module, the second dental provider with access to the EDR file associated with the patient account; and
schedule, by a scheduling module, an appointment for the patient for a recommended treatment with the second dental provider.

2. The system of claim 1, wherein:
the indication corresponding to the digital file comprises information associated with an email account associated with the dental patient; and
obtaining the digital file comprises obtaining the digital file from the email received by the email account.

3. The system of claim 2 wherein obtaining the digital file from the email received by the email account comprises obtaining the digital file from an attachment of the email.

4. The system of claim 2 wherein the at least one processor is further configured to:
receive a request to set up the patient account from the patient device; and
set up the patient account based at least in part on the request, the setting up of the patient account comprising assigning the email account to the patient account.

5. The system of claim 1, wherein:
the indication corresponding to the digital file comprises a location of the digital file on the patient device; and
obtaining the digital file comprises uploading the digital file from the location of the digital file on the patient device.

6. The system of claim 1, wherein:
the indication corresponding to the digital file comprises an indication of the performance of an image capture operation by the patient device; and
obtaining the digital file comprises obtaining the digital file from the image capture operation performed by the patient device.

7. The system of claim 1 wherein providing the second dental provider with access to the EDR file associated with the patient account comprises attaching the EDR file associated with the patient account to an email.

8. The system of claim 1 wherein providing the second dental provider with access to the EDR file associated with the patient account comprises generating a link to the EDR file associated with the patient account, the link being usable by the second dental provider to view the EDR file associated with the patient account.

9. The system of claim 1 wherein the at least one processor is further configured to:
identify a plurality of dental providers that meet a set of criteria corresponding to the dental patient, the plurality of dental providers comprising the second dental provider;
request estimates for dental services from the identified dental providers;
obtain estimates from at least one of the identified dental providers including an estimate from the second dental provider;
cause presentation of the obtained estimates on the user interface of the patient device; and
obtain, from the patient device, a selection of the estimate obtained from the second dental provider.

10. The system of claim 9 wherein:
receiving, from the patient device, information identifying the second dental provider comprises receiving the set of criteria from the patient device; and
providing the second dental provider with access to the EDR file associated with the patient account comprises providing the second dental provider with access to the EDR file associated with the patient account based at least in part on the request of the estimate for dental services from the second dental provider.

11. The system of claim 9 wherein the at least one processor is further configured to:
obtain an indication of a schedule of available appointments corresponding to the second dental provider;
cause the user interface to present the indication on the patient device;
obtain, from the patient device, a selection of an available appointment from the schedule; and
cause a communication with a provider device associated with the second dental provider that indicates that the dental patient has selected the available appointment from the schedule.

12. A method performed by at least one processor comprising hardware, the method comprising:
maintaining, by an account module, a patient account associated with a dental patient;
generate, by a user interface module, a user interface to be presented on a patient device associated with the dental patient;
receiving, by a computing system, from the patient device, an indication corresponding to a digital file associated with the dental patient that was generated by a first dental provider;
obtaining, by a capture module, based on the received indication, the digital file;
processing and categorizing the digital file using artificial intelligence (AI) and pattern matching;
generating, by the capture module, an electronic dental record (EDR) file based at least in part on the processed digital file;
storing, by the capture module, the EDR file in an EDR database;
associating, by the account module, the EDR file with the patient account of the dental patient;
recommending, by a recommendation module, a patient treatment plan based on the EDR file, the patient treatment plan comprising at least a list of recommended dental providers and treatments;
receiving, from the patient device, information identifying a second dental provider from the list of recommended dental providers;
providing, by an export module, the second dental provider with access to the EDR file associated with the patient account; and
schedule, by a scheduling module, an appointment for the patient for a recommended treatment with the second dental provider.

13. The method of claim 12, wherein:
the indication corresponding to the digital file comprises information associated with an email account associated with the dental patient; and
obtaining the digital file comprises obtaining the digital file from the email received by the email account.

14. The method of claim 13 wherein method further comprises:
receiving a request to set up the patient account from the patient device; and
setting up the patient account based at least in part on the request, the setting up of the patient account comprising assigning the email account to the patient account.

15. The method of claim 12 wherein providing the second dental provider with access to the EDR file associated with the patient account comprises attaching the EDR file associated with the patient account to an email.

16. The method of claim 12 wherein the method further comprises:
identifying a plurality of dental providers that meet a set of criteria corresponding to the dental patient, the plurality of dental providers comprising the second dental provider;
requesting estimates for dental services from the identified dental providers;
obtaining estimates from at least one of the identified dental providers including an estimate from the second dental provider;
causing presentation of the obtained estimates on the user interface of the patient device; and
obtaining, from the patient device, a selection of the estimate obtained from the second dental provider.

17. The method of claim 16 wherein:
receiving, from the patient device, information identifying the second dental provider comprises receiving the set of criteria from the patient device; and
providing the second dental provider with access to the EDR file associated with the patient account comprises providing the second dental provider with access to the EDR file associated with the patient account based at least in part on the request of the estimate for dental services from the second dental provider.

18. A non-transitory computer-readable medium storing instructions that, when executed by at least one processor, cause the at least one processor to:
maintain, by an account module, a patient account associated with a dental patient;
generate, by a user interface module, a user interface to be presented on a patient device associated with the dental patient;
receive, by a computing system, from the patient device, an indication corresponding to a digital file associated with the dental patient that was generated by a first dental provider;
obtain, by a capture module, based on the received indication, the digital file;
process and categorize the digital file using artificial intelligence (AI) and pattern matching;
generate, by the capture module, an electronic dental record (EDR) file based at least in part on the processed digital file;
store, by the capture module, the EDR file in an EDR database;
associate, by the account module, the EDR file with the patient account of the dental patient;
recommend, by a recommendation module, a patient treatment plan based on the EDR file, the patient treatment plan comprising at least a list of recommended dental providers and treatments;
receive, from the patient device, information identifying a second dental provider from the list of recommended dental providers;
provide, by an export module, the second dental provider with access to the EDR file associated with the patient account; and
schedule, by a scheduling module, an appointment for the patient for a recommended treatment with the second dental provider.

19. The non-transitory computer-readable medium of claim 18, wherein:
the indication corresponding to the digital file comprises information associated with an email account associated with the dental patient; and
obtaining the digital file comprises obtaining the digital file from the email received by the email account.

20. The non-transitory computer-readable medium of claim 18 wherein providing the second dental provider with access to the EDR file associated with the patient account comprises attaching the EDR file associated with the patient account to an email.
